SEQUENCE LISTING (3 sequences)

SEQ ID NO: 1 - DNA, 12 bp, Artificial
  synthetic
atttatgttttc

SEQ ID NO: 2 - DNA, 33 bp, Artificial
  synthetic
atttatgtgtgtgtgtgtgtgtgtgtgtttttc

SEQ ID NO: 3 - DNA, 31 bp, Artificial
  synthetic
aacacacacacacacacacacacatatatgt

(19) United States
(12) Patent Application Publication
(10) Pub. No.: US 2016/0040254 A1
(43) Pub. Date: Feb. 11, 2016

(54) PROGNOSTIC AND DIAGNOSTIC METHODS BASED ON QUANTIFICATION OF SOMATIC MICROSATELLITE VARIABILITY

(71) Applicants: Harold Garner, Blacksburg, VA (US); Zalman Vaksman, Blacksburg, VA (US)

(72) Inventors: Harold Garner, Blacksburg, VA (US); Zalman Vaksman, Blacksburg, VA (US)

(73) Assignee: VIRGINIA TECH INTELLECTUAL PROPERTIES, INC., Blacksburg, VA (US)

(21) Appl. No.: 14/815,050

(22) Filed: Jul. 31, 2015

Related U.S. Application Data
(60) Provisional application No. 62/031,461, filed on Jul. 31, 2014.

(51) Int. Cl. C12Q 1/68

(52) U.S. Cl. CPC C12Q 1/6886; C12Q 1/6883; C12Q 2600/158; C12Q 2600/156; C12Q 2600/118; C12Q 2600/166

(57) ABSTRACT

Prognostic and diagnostic methods based on somatic microsatellite variability measurements are described. Somatic microsatellite variability measurements can be used to identify defects in DNA repair pathways in a biological sample and to predict or identify one or more pathologies. The methods may statistically compare somatic microsatellite variability between a sample from a cell line or subject with that of a reference sample with defined pathological characteristics and/or defined lesions in DNA repair pathways. The methods are useful for determining the probability that an individual may develop a pathology, such as a cancer, within a specific time frame, and/or are useful as a diagnostic test. The methods have clinical utility in that somatic variability can be evaluated and/or measured as an indication of the amount of genomic instability (e.g., microsatellite and non-microsatellite/SNP), which can then be used as a clinical diagnostic to make clinical decisions, especially in the area of cancer.

### CROSS-REFERENCE TO RELATED APPLICATIONS

The present application relies on the disclosure of and claims priority to and the benefit of the filing date of U.S. Provisional Application No. 62/031,461, filed Jul. 31, 2014, the disclosure of which is hereby incorporated by reference herein in its entirety.

### BACKGROUND OF THE INVENTION

1. Field of the Invention

The present invention relates to the field of molecular prognostics and diagnostics. More particularly, the present invention relates to the use of somatic microsatellite variability measurements to identify specific defects in DNA repair pathways in a biological sample and to predict or identify one or more pathologies based on these measurements.

2. Description of Related Art

Microsatellites (MSTs) are regions of repetitive DNA at which 1-6 nucleotides are tandemly repeated, and are present ubiquitously throughout the genome, both in gene and intergenic regions. Observations of somatic variation in MSTs have demonstrated that MST mutation rates are between 10 and 1000 times higher than that of surrounding DNA (Gemayel R, Vinces M D, Legendre M, Verstrepen K J (2010) Variable tandem repeats accelerate evolution of coding and regulatory sequences. Annu Rev Genet 44: 445-477, 2; and Fonville N C, Ward R M, Mittelman D (2011) Stress-induced modulators of repeat instability and genome evolution. J Mol Microbiol Biotechnol 21: 36-44), rendering microsatellites mutational “hot-spots” (Bagshaw A T, Pitt J P, Gemmell N J (2008) High frequency of microsatellites in S. cerevisiae meiotic recombination hotspots. BMC Genomics 9: 49; and Payseur B A, Jing P, Haasl R J (2011) A genomic portrait of human microsatellite variation. Mol Biol Evol 28: 303-312). The increased mutational rate of MSTs is thought to be primarily due to DNA polymerase slippage and misalignment of the slipped structure due to local homology (Delagoutte E, Goellner G M, Guo J, Baldacci G, McMurray C T (2008) Single-stranded DNA-binding protein in vitro eliminates the orientation-dependent impediment to polymerase passage on CAG/CTG repeats. J Biol Chem 283: 13341-13356; Hile S E, Eckert K A (2008) DNA polymerase kappa produces interrupted mutations and displays polar pausing within mononucleotide microsatellite sequences. Nucleic Acids Res 36: 688-696; and Ananda G, Walsh E, Jacob K D, Krasilnikova M, Eckert K A, et al. (2013) Distinct mutational behaviors differentiate short tandem repeats from microsatellites in the human genome. Genome Biol Evol 5: 606-620). This difference in primary mutational mechanism suggests that, unlike non-repetitive DNA whose mutational spectrum is primarily SNPs, microsatellites are more prone to INDELs (Payseur B A, Jing P, Haasl R J (2011) A genomic portrait of human microsatellite variation. Mol Biol Evol 28: 303-312; Ananda G, Walsh E, Jacob K D, Krasilnikova M, Eckert K A, et al. (2013) Distinct mutational behaviors differentiate short tandem repeats from microsatellites in the human genome. Genome Biol Evol 5: 606-620; and Leclercq S, Rivals E, Jarne P (2010) DNA slippage occurs at microsatellite loci without minimal threshold length in humans: a comparative genomic approach. Genome Biol Evol 2: 325-335). Specifically MSTs are prone to INDELs that are ‘in-phase’ or result in expansion or contraction by complete repeat units. For example, a dimer microsatellite can typically expand or contract by 2N nucleotides while a trimer can expand or contract by 3N (Gemayel R, Vinces M D, Legendre M, Verstrepen K J (2010) Variable tandem repeats accelerate evolution of coding and regulatory sequences. Annu Rev Genet 44: 445-477). MSTs are found in and around a significant number of coding and promoter regions and specific microsatellite variations have been linked to over 40 disorders, such as the CAG microsatellite whose expansion is associated with Huntington's disease and the CGG repeat whose expansion is associated with Fragile X (Gemayel R, Vinces M D, Legendre M, Verstrepen K J (2010) Variable tandem repeats accelerate evolution of coding and regulatory sequences. Annu Rev Genet 44: 445-477; and Budworth H, McMurray C T (2013) Bidirectional transcription of trinucleotide repeats: roles for excision repair. DNA Repair (Amst) 12: 672-684). In addition, a more general increase in MST instability has been associated with colon cancer, which, if detected, results in better prognosis and can influence treatment (Xiao H, Yoon Y S, Hong S M, Roh S A, Cho D H, et al. (2013) Poorly differentiated colorectal cancers: correlation of microsatellite instability with clinicopathologic features and survival. Am J Clin Pathol 140: 341-347; and Hong S P, Min B S, Kim T I, Cheon J H, Kim N K, et al. (2012) The differential impact of microsatellite instability as a marker of prognosis and tumour response between colon cancer and rectal cancer. Eur J Cancer 48: 1235-1243). Currently, MST instability is clinically defined based on the results of a kit that tests somatic variation of 18-21 “susceptible” loci (PowerPlex®21, Promega). Although the test has been shown to be effective for identifying MST unstable colon cancer (Barber L J, Rosa Rosa J M, Kozarewa I, Fenwick K, Assiotis I, et al. (2011) Comprehensive genomic analysis of a BRCA2 deficient human pancreatic cancer. PLoS One 6: e21639), it is significantly less effective for most other disorders including other cancers (Lacroix-Triki M, Lambros M B, Geyer F C, Suarez P H, Reis-Filho J S, et al. (2010) Absence of microsatellite instability in mucinous carcinomas of the breast. Int J Clin Exp Pathol 4: 22-31; Yoon K, Lee S, Han T S, Moon S Y, Yun S M, et al. (2013) Comprehensive genome- and transcriptome-wide analyses of mutations associated with microsatellite instability in Korean gastric cancers. Genome Res 23: 1109-1117; and Kim T M, Laird P W, Park P J (2013) The landscape of microsatellite instability in colorectal and endometrial cancer genomes. Cell 155: 858-868). Thus, there is still a need to capture and discern variation patterns which would provide a more accurate and useful clinical data for a broader range of disorders.

### SUMMARY OF THE INVENTION

Embodiments of the invention provide for prognostic and diagnostic methods based on somatic microsatellite variability measurements. The somatic microsatellite variability measurements can be used to identify specific defects in DNA repair pathways in a biological sample and to predict the occurrence of or identify one or more pathologies based on these measurements. The methods may statistically compare the somatic microsatellite variability measurements or data between a sample from a cell line or a subject with corresponding measurements from one or more reference samples with defined pathological characteristics and/or defined lesions in DNA repair pathways. In addition, the methods of the invention may be used to measure somatic variability as a gauge of the amount of genomic instability (microsatellite and non-microsatellite/SNP) in a clinical sample which can then be used as a clinical diagnostic to make clinical decisions for treating a disease, such as cancer. The amount of genomic instability may serve as a biomarker for clinical characteristics of the cancer and can provide an indication of cancer prognosis and inform treatment decisions.

### BRIEF DESCRIPTION OF THE DRAWINGS

The accompanying drawings illustrate certain aspects of embodiments of the present invention, and should not be used to limit the invention. Together with the written description the drawings serve to explain certain principles of the invention.

FIG. 1 is a Table showing a list of commonly studied DNA repair deficiency disorders, the genes affected, cancer predisposition and age of onset for cancer. (Abbreviations: HR—Homologous Recombination, CLR—Crosslink Repair, NHEJ—Nonhomologous End Joining, MMR—Mismatch Repair, BER—Base Excision Repair, TC—transcriptional)

FIG. 2A is a chart that shows a Sanger sequencing output (locus is shown in the first 5 columns in line 1) predict 3 different length alleles. The major is 23 nts with 2 minor alleles, 25 and 21 nts long.

FIG. 2B is a graph that shows a Sanger sequencing chromatogram. The black arrows are showing the start point of different alleles.

FIG. 3 is a Table showing MST and non-MST from standard exome sequencing of ‘normal’ cells, but not from sequencing of a single cell after whole genome amplification, show the expected high ratio of INDELs (expansions and contractions) to SNPs.

FIG. 4 is a Table showing the percent of heterozygotic and homozygotic loci as well as the percent of loci with minor alleles, for each sample or cell line. # indicates significantly different from normal cell mean p<0.01.

FIG. 5 is a Table showing percent of SNPs and INDELs differ in DNA repair defective cell lines compared to “normal” cells. # indicates significantly different from normal cell mean p<0.01.

FIGS. 6A and 6B are graphs showing effects of sequencing error and the minimum number of reads required to call an allele on the number of alleles called in sequencing data. Modeling data with different error frequencies (0.5%-5%) showed an increase in loci with multiple alleles as error increased when both 2 (FIG. 6A) and 3 (FIG. 6B) reads were minimally required to call an allele. In contrast, standard exome sequencing data from DNA repair proficient cells (PD20 RV:D2 cells) and exome sequencing after whole genome amplification from a single cell were insensitive to the cut-off used.

FIGS. 7A and 7B are graphs showing variation in average depth per locus cannot explain the number of loci with minor alleles. The average read depth at loci with increasing numbers of alleles using (FIG. 7A) 2 and (FIG. 7B) 3 confirming reads per allele for in-silico generated data using 1% and 2.5% induced error rate for 4 different cell lines.

FIG. 8A is a graph showing DNA repair proficient cells vary significantly from the in-silico modeling and single cell sequencing analysis with respect to SNPs and INDELs. The percent of SNPs, expansion and contractions for single cell sequencing and the in-silico model as well as the mean and standard deviation for the control cell lines. * significant difference p<0.01.

FIG. 8B is a Table corresponding to the data in FIG. 8A.

FIG. 9 is a Table showing exome sequencing data which indicates that MST and non-MST haplotype and somatic polymorphism are reproducible in DNA repair proficient cell lines.

FIG. 10 is a Table showing MST and non-MST containing loci from exome sequencing of DNA repair proficient cells, but not from sequencing of a single cell after whole genome amplification, show the expected high ratio of INDELs (expansions and contractions) to SNPs.

FIG. 11 is a Table showing Percent concordance/discordance of haplotype and loci with minor alleles for cell lines.

FIGS. 12A-12D are graphs showing a regression analysis indicates a significant within and between cell line correlation in the fraction of loci with one or more minor alleles. Full factorial plots of the fraction of loci with minor alleles by chromosome, regression line and correlation coefficient for (FIG. 12A) PD20 RV:D2-1 and (FIG. 12C) PD20 RV:D2-1, 2, MCF10A and HEK293. Also full factorial plots of the fraction of loci with minor alleles for the corresponding 1 million base segments of all the chromosomes, a regression line and the correlation coefficient for (FIG. 12B) PD20 RV:D2-1 and 2 (FIG. 12D) PD20 RV:D2-1, 2, MCF10A and HEK293.

FIG. 13 is a Table showing haplotype distribution and somatic polymorphism rate differ in DNA repair defective cell lines compared to DNA repair proficient cell lines.

FIG. 14 is a Table showing SNP and INDEL fractions differ in DNA repair defective cell lines compared to DNA repair proficient cells.

FIG. 15 is a graph and chromosome image showing the distribution of MST loci showing somatic variability for chromosome 1 binned into 1 million base regions in PD20 and the derived PD20 RV:D2 cell line. The horizontal line demarcates outlier segments, based on a X² distribution. All genes shown were found to contain exonal MSTs that with at least 2 minor alleles in both PD20 RV:D2 samples and were found in regions that exceeded the demarcated level. Genes shown in red were found to contain exonal MSTs with at least 2 minor alleles in all 4 DNA repair proficient cell line samples and those shown in blue were found in 3 of the 4 samples. The chromosome image shown at the bottom was obtained from Wikipedia.

FIGS. 16A and 16B are graphs showing an increase in the fraction of reads substantiating the second alleles if present, and all minor alleles. The average fraction of reads representing (FIG. 16A) all minor alleles (only for loci with minor alleles) and (FIG. 16B) the second allele in both heterozygotic and homozygotic loci that have at least one minor allele, for DLD-1, PD20 and Capan-1 cells were compared to the average of the DNA repair proficient cell lines. The (+) denotes a significant difference from DNA repair proficient (p<0.01) with z-test.

FIGS. 17A and 17B are graphs showing a comparison of the percent of heterozygotic loci and loci exhibiting SMV in exons and untranslated genomic regions in DNA repair proficient and impaired cell lines. FIG. 17A shows the percent of MST loci that for which minor alleles were found and FIG. 17B shows the percent of heterozygotic MST loci, in exons and untranslated regions. Depicted in both figures are the means for the DNA repair proficient cell lines and the individual percentage for PD20, DLD-1 and Capan-1 cell lines. (+) p<0.05 as compared to DNA proficient cells and (*) p<0.001 as compared to DNA proficient cells in measurement of the difference between exons and untranslated regions.

FIGS. 18A and 18B are pie charts showing the distribution of genes that show SMV in DNA repair deficient cell lines appears random while those in the DNA repair proficient cell lines show significant similarity. The percent of genes with MSTs that with MSTs that have a minimum of 2 minor alleles in (FIG. 18A) DNA repair proficient cell lines and (FIG. 18B) DNA repair deficient cell lines that are found in all of the or some of the sequenced samples. In FIG. 18A, the genes that are present in all three DNA repair deficient cell lines is 0.3% and the slice of the pie chart is not visible due to the small percentage.

### DETAILED DESCRIPTION OF VARIOUS EMBODIMENTS OF THE INVENTION

Reference can now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

Embodiments of the invention comprise prognostic and diagnostic methods for identifying DNA repair deficiencies and associated clinical pathologies.

One embodiment of the invention provides a method of detecting a pathological feature in a subject, comprising taking a biological sample, wherein the biological sample comprises one or more cells of the subject, subjecting the biological sample to DNA sequencing to determine a pattern of somatic microsatellite variability in the sample, and comparing a pattern of somatic microsatellite variability in the sample with a pattern of microsatellite variability of one or more reference samples to identify one or more pathological features in the one or more cells of the subject.

Another embodiment of the invention provides a method of detecting a pathological feature in a subject, the method comprising taking a biological sample, wherein the biological sample comprises one or more cells of the subject, subjecting the biological sample to DNA sequencing to determine one or more somatic microsatellite variability measurements in the sample, and statistically comparing the one or more somatic microsatellite variability measurements in the sample with one or more somatic microsatellite variability measurements of one or more reference samples to identify one or more pathological features in the one or more cells of the subject. In embodiments, the one or more somatic microsatellite variability measurements are selected from the group consisting of percentage of SNPs, percentage of expansions, percentage of contractions, ratio of expansions and contractions to SNPs, percentage of heterozygotic loci, percentage of homozygotic loci, and percentage of loci with minor alleles.

Methods of the invention also include a method of treating a patient, comprising: taking a clinical sample from a patient, wherein the clinical sample comprises one or more cells; subjecting the clinical sample to DNA sequencing to determine one or more somatic variability measurements in the clinical sample; and statistically comparing the one or more somatic variability measurements in the clinical sample with one or more somatic variability measurements of one or more reference samples to interpret an amount of genomic instability in the clinical sample; and making a treatment decision based on the interpretation; optionally wherein the one or more somatic variability measurements are selected from the group consisting of percentage of SNPs, percentage of expansions, percentage of contractions, ratio of expansions and contractions to SNPs, percentage of heterozygotic loci, percentage of homozygotic loci, and percentage of loci with minor alleles. Such methods are useful for treating cancer, including colon cancer. Included are such methods, wherein the clinical sample is blood and wherein the treatment decision concerns predicting risk of cancer, or other disease of the aged, or cardiac disease, or neurological disease.

In some embodiments, the biological sample is taken from a cell line. In other embodiments, the biological sample is a clinical sample from a patient. In some embodiments, the one or more pathological features are one or more deficient DNA repair pathways. The one or more reference samples may be obtained from one or more cells with proficient DNA repair capability or from one or more cells with one or more deficient DNA repair pathways. The one or more deficient DNA repair pathways may include homologous recombination, non-homologous end joining, DNA mismatch repair, base excision repair, nucleotide excision repair, and crosslink repair. Further, the one or more pathological features may indicate a genetic syndrome in the patient such as Bloom Syndrome, Rothmund-Thomson, Fanconi Anemia, Werner Syndrome, BRCA1/2 mutation, Lynch Syndrome, Cockayne Syndrome, Xeroderma Pigmetnosum, and Trichothiodystropia. Additionally, the one or more pathological features indicate a cancer in the patient. The DNA sequencing may be performed on the whole genome or whole exome of the one or more cells or a partial genome or exome.

In some embodiments, a sample such as a biopsy or blood sample comprising one or more cells is taken from an individual and is subject to DNA sequencing analysis, which is used to identify one or more somatic microsatellite variability patterns in the cells. The somatic microsatellite variability patterns can be used to identify specific patterns or signatures of defective DNA repair mechanisms in the cells. In addition, in embodiments the somatic microsatellite variability patterns can be used to identify or predict the occurrence of one or more diseases associated with the defective DNA repair mechanisms, including cancer, neurological diseases, and cardiac diseases.

Biopsy procedures for taking samples of cells from a subject are known. Examples include bone marrow biopsies, endoscopic biopsies, and needle biopsy procedures including fine needle aspiration, core needle biopsies, vacuum-assisted biopsies, and image-guided biopsies. These can be performed according to standard protocols used when taking a biopsy for pathology. Optionally, a portion of the tissue sample can either be immediately put in culture or cryopreserved with the use of a cryoprotectant, such as DMSO or glycerol, for later culturing or DNA sequencing analysis. Thus, the biopsy procedures need not be elaborated here. In some cases, multiple biopsies may be warranted. The biopsy samples may be taken from healthy or diseased tissue, such as a cancer. In some embodiments, the tissue that is the subject of a biopsy appears healthy on a macroscopic level yet contains one or more lesions on a molecular level. The one or more lesions may include deficiencies in DNA repair enzymes.

In some embodiments, the biopsy is taken from a tumor. The tumor that may be sampled may be from a cancer selected from the group consisting of brain cancer, breast cancer, colon cancer, rectal cancer, endometrial cancer, cervical cancer, kidney cancer, leukemia, liver cancer, stomach cancer, esophageal cancer, oral cancer, throat cancer, tracheal cancer, lung cancer, melanoma, non-melanoma skin cancers, non-Hodgkin lymphoma, Hodgkin lymphoma, pancreatic cancer, prostate cancer, head and neck cancers, bone cancer, and thyroid cancer.

In some embodiments, a sample of cells is taken from saliva, skin, or hair. In some embodiments, a sample of cells is taken from a peripheral blood sample (through standard phlebotomy techniques), a urine sample, a fecal sample, or a cerebrospinal fluid sample. In some embodiments, the sample of cells is taken from an organ or tissue such as liver, kidney, pancreas, heart, lungs, spleen, small intestine, large intestine, stomach, colon, gall bladder, bladder, lymph nodes, brain, or nerves. In some embodiments, a sample of cells is taken from endocrine, exocrine, or nervous tissue.

The cells from the portion of the biopsy may be cultured through a variety of methods known for tissue culture, primary cell culture, and cancer cell culture. For example, for primary cell culture, the tissue sample may be first dissected to remove fatty and necrotic cells. Then, the tissue sample may be subject to enzymatic or mechanical disaggregation. The dispersed cells may then be incubated, and the media changed to remove loose debris and unattached cells. Because primary cells are anchorage-dependent, adherent cells, they require a surface in order to grow properly in vitro. In one embodiment, the cells are cultured in two-dimensional (2D) cultures. Typically, a plastic uncoated vessel such as a flask or petri dish is used, and the cells are bathed in a complete cell culture media, composed of a basal medium supplemented with appropriate growth factors and cytokines. During establishment of primary cultures, it may be useful to include an antibiotic in the growth medium to inhibit contamination introduced from the host tissue. Various protocols for culturing primary cells are known and a variety of resources are available, including the ATCC® Primary Cell Culture Guide, available on the American Type Culture Collection (ATCC) website, Human Cell Culture Protocols (Methods in Molecular Biology), Mitry, Ragai R., and Hughes, Robin D. (Eds.), 2012, and Cancer Cell Culture: Methods and Protocols (Methods in Molecular Biology) Ian A. Cree (Ed.), 2011.

Next, a direct biopsy sample, cryopreserved biopsy sample, or a cultured sample can be subject to sequencing analysis. Various sequencing approaches are known, including Sanger (or dideoxy) method, Maxam-Gilbert, Primer Walking, and Shotgun Sequencing. Preferred are next-generation sequencing methods (also known as high-throughput sequencing), which include a number of different sequencing methods including Illumina (Solexa) sequencing, Roche 454 sequencing, Ion torrent: Proton/PGM sequencing, and SOLiD sequencing. Such next-generation techniques have been reviewed in the literature (see Grada and Weinbrecht, Next-Generation Sequencing: Methodology and Application Journal of Investigative Dermatology (2013) 133, e11; and Bahassi and Stambrook, Next-generation sequencing technologies: breaking the sound barrier of human genetics, Mutagenesis, 2014 Sep.; 29(5):303-10). Next generation sequence methods may encompass whole genome, whole exome, and partial genome or exome sequencing methods. Whole exome sequencing covers the protein-coding regions of the genome, which represents just over 1% of the genome. Specific protocols for whole exome sequencing are provided in Example 1 and Example 2.

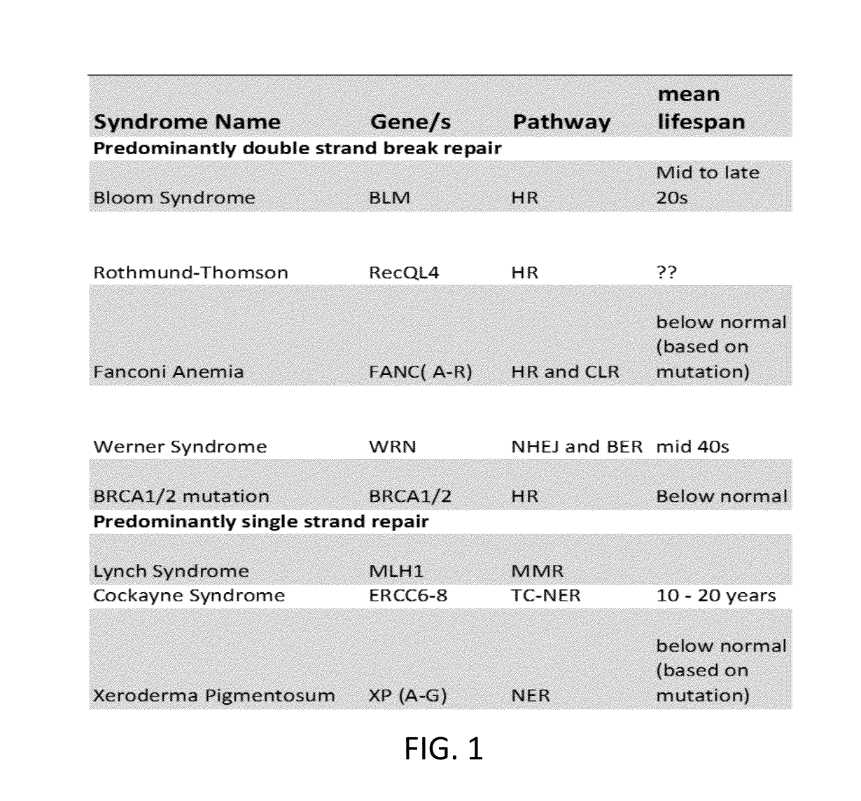

From the results of sequencing, patterns of somatic microsatellite variation can be obtained. The patterns of somatic microsatellite variation can be represented in a variety of measurements, including percentage of SNPs, expansions, contractions, or ratio of INDELS (expansions and contractions) to SNPs. The patterns of somatic microsatellite variation can also be represented as haplotype measurements such as percent of heterozygotic and homozygotic loci as well as percent of loci with minor alleles. Based on these measurements, specific patterns associated with both normal DNA repair capability and impaired DNA capability can be obtained, and these patterns may also associate with specific defect DNA repair mechanisms/pathways and their associated pathologies, such as the syndromes shown in the Table in FIG. 1. Such patterns are shown in the tables and figures corresponding with the following Examples, which further illustrate embodiments of the invention.

Further, in some embodiments, the patterns of microsatellite variation described above may be used as a basis for statistical comparisons between samples of defined clinical, subclinical, or molecular characters and samples of unknown characteristics. For example, in one embodiment, a sample of one or more cells is taken from a subject for which a diagnosis or characterization is sought. The one or more cells of the subject are analyzed via DNA sequencing analysis to determine the patterns of somatic microsatellite variation as represented by the measurements described above. Then, a statistical comparison can be made with samples of defined clinical, subclinical, or molecular characteristics, which may include the presence of defects in DNA repair pathways, the presence of a progeroid disorder, the presence of a disease such as a cancer, etc., wherein such comparison is based on measurements or data including percentage of SNPs, expansions, contractions, or ratio of INDELS (expansions and contractions) to SNPs, or haplotype measurements such as percent of heterozygotic and homozygotic loci as well as percent of loci with minor alleles.

In some embodiments, the patterns of microsatellite variation may indicate the presence of a cancer. For example, the sample of a subject may be statistically compared with one or more reference samples having a known cancer diagnosis which correlate with specific patterns of somatic microsatellite variation, and from such statistical comparison a specific cancer may be diagnosed in the subject, or the subject may be determined to be free of the cancer at least in the specific sample.

Additionally, in some embodiments, the patterns of microsatellite variation may be used to indicate one or more characteristics of a cancer, including whether benign or malignant, aggressiveness, stage of cancer, and grade of tumor. For example, the sample of a subject may be statistically compared with one or more reference samples having known cancer cell characteristics which correlate with specific patterns of somatic microsatellite variation, and from such statistical comparison, the aggressiveness, stage of cancer, and grade of tumor may be diagnosed in the subject.

Additionally, in some embodiments, the patterns of microsatellite variation may be used to indicate one or more characteristics of other disorders, and accelerated aging or progeroid disorders, which may include without limitation Ataxia telangiectasia, Bloom syndrome, Cockayne's syndrome, Fanconi's anaemia, Progeria (Hutchinson-Gilford Progeria syndrome), Rothmund-Thomson syndrome, Trichothiodystrophy, Werner syndrome, and Xeroderma pigmentosum. For example, the sample of a subject may be statistically compared with one or more reference samples representing known progeroid disorders which correlate with specific patterns of somatic microsatellite variation, and from such statistical comparison, a progeroid disorder may be diagnosed in the subject.

Statistical comparisons and tests are known in the art. These include basic statistical analyses such as the t-test, z-test, ANOVA, and correlation. Further, in some embodiments, machine learning algorithms can be used as the basis to predict or classify samples of unknown characteristics based on samples of known characteristics, using the somatic microsatellite variability data as a basis for such predictions or classifications. Such machine learning algorithms may include without limitation hierarchical clustering, k-means clustering, linear discriminant analysis, principle components analysis, logistic regression, support vector machines, k-nearest neighbor, decision trees, neural networks, Bayesian networks, and Hidden Markov models. Such statistical comparisons can be applied to the data in the Tables in the following Examples.

Embodiments of the methods of the invention may include both prognostic and diagnostic determinations. Prognostic determinations provide a probability or likelihood of development of a disease in the future. Diagnostic determinations provide a probability or likelihood of the presence of a disease in the present time. For example, in some prognostic embodiments, the methods of the invention may be used to determine the probability that an individual may develop a disease such as a cancer within a specific time frame. Based on such prognostic assessment, the individual may take action such as modification of life style or other risk factors to forestall development of the pathology. In some diagnostic embodiments, the methods of the invention may be used as a diagnostic test for the presence of a particular disease. In some embodiments, the methods of the invention may be used to supplement or as an alternative to standard cellular and molecular pathology measurements for making treatment decisions based on clinical samples, such as a biopsy of a cancer. For example, the methods of the invention may provide measurements which indicate the amount of genomic instability in a cancer, which may be used to classify tumors and inform and optimize the type of treatment such as radiation, type of chemotherapy, dosage, etc. and/or provide a prognosis for the patient. In some prognostic embodiments, the level of genomic stability in a subject may indicate a need for nutritional or other lifestyle intervention to prevent a disease such as cancer. For example, a patient's colon biopsy may indicate the likelihood of developing various forms of colon cancer based on the level of genomic instability interpreted from the methods of the invention, and a clinician may recommend specific therapeutic regimens or dietary changes based on the interpretation. For both prognostic and diagnostic embodiments, the tests may provide measurements of accuracy including sensitivity, specificity, false positives, false negatives, and geometric mean.

Embodiments of the invention may include the development of a library of somatic microsatellite variability data that may be used in the methods of the invention. The library of microsatellite variability data may be derived from experiments with cell lines (such as those described in the Examples), clinical pathology samples, such as archived specimens or fresh pathology samples, and the like. The library of the somatic microsatellite variability data may be used as reference samples for performing the prognostic and diagnostic methods of the invention. The library of somatic microsatellite variability data may be stored in a non-transitory computer readable medium, such as a database.

Embodiments of the invention may also include a set of computer executable instructions for carrying out the statistical comparisons or performing the algorithms of the invention. The computer-executable instructions may be organized into routines, subroutines, procedures, objects, methods, functions, or any other organization of computer-executable instructions that is known or becomes known to a skilled artisan in light of this disclosure, where the computer-executable instructions are configured to direct a computer or other data processing device such as a processor to perform one or more of the specified processes and operations. The computer-executable instructions may be written in any suitable programming language and may be stored on a non-transitory computer readable medium, such as in the memory of a computer.

Embodiments of the invention also include a non-transitory computer readable medium comprising one or more computer files comprising a set of computer-executable instructions for performing one or more of the calculations, steps, processes and operations described and/or depicted herein. In exemplary embodiments, the files may be stored contiguously or non-contiguously on the computer-readable medium. Embodiments may include a computer program product comprising the computer files, either in the form of the computer-readable medium comprising the computer files and, optionally, made available to a consumer through packaging, or alternatively made available to a consumer through electronic distribution. As used in the context of this specification, a “computer-readable medium” includes any kind of computer memory such as floppy disks, conventional hard disks, CD-ROM, Flash ROM, non-volatile ROM, electrically erasable programmable read-only memory (EEPROM), and RAM.

In other embodiments of the invention, files comprising the set of computer-executable instructions may be stored in computer-readable memory on a single computer or distributed across multiple computers. A skilled artisan will further appreciate, in light of this disclosure, how the invention can be implemented, in addition to software, using hardware or firmware. As such, as used herein, the operations of the invention can be implemented in a system comprising any combination of software, hardware, or firmware.

Embodiments of the invention include one or more computers or devices loaded with a set of the computer-executable instructions described herein. The computers or devices may be a general purpose computer, a special-purpose computer, or other programmable data processing apparatus to produce a particular machine, such that the one or more computers or devices are instructed and configured to carry out the calculations, processes, steps, and operations of the invention. The computer or device performing the specified calculations, processes, steps, and operations may comprise at least one processing element such as a central processing unit (i.e. processor) and a form of computer-readable memory which may include random-access memory (RAM) or read-only memory (ROM). The computer-executable instructions can be embedded in computer hardware or stored in the computer-readable memory such that the computer or device may be directed to perform one or more of the processes and operations depicted and/or described herein.

Additional embodiments of the invention comprise a computer system for carrying out methods of the invention or steps of the method requiring computer implementation. The computer system may comprise a processor for executing the computer-executable instructions, one or more databases described herein, a user interface, and a set of instructions (e.g. software) for carrying out the method. The computer system can be a stand-alone computer, such as a desktop computer, a portable computer, such as a tablet, laptop, PDA, or smartphone, or a set of computers connected through a network including a client-server configuration and one or more database servers. The network may use any suitable network protocol, including IP, UDP, or ICMP, and may be any suitable wired or wireless network including any local area network, wide area network, Internet network, telecommunications network, Wi-Fi enabled network, or Bluetooth enabled network.

### EXAMPLES

The capabilities described in the following Examples specifically address the issue of genomic stability by observing changes in somatic variability in MSTs and in non-repetitive DNA. The Examples show that the pattern of somatic MST variability will be the signature for the specific defective DNA repair pathway in DNA Repair Deficiency Disorders (DRDDs). The known DRDDs are used to confirm this relationship as the Examples show that MST variability can differentiate between cell lines with known defects in various DNA repair mechanisms (e.g. mismatch repair, DNA crosslink repair, homologous recombination), which correlate with an altered distribution of loci with non-haplotype alleles. The findings of the Examples indicate that signatures that distinctly define specific defective DNA repair mechanisms can be gleaned from next-generation sequencing data and that this information may be used as a diagnostic or prognostic tool to identify individuals with altered levels of somatic variation that may indicate the presence of or increased risk for disease such as cancer, or the evaluation of patient's tumor that may yield clinically actionable information.

### Example 1

DNA sequencing: Preliminary sequencing data and analysis methods were obtained using the methods listed below.

Exome paired-end libraries can be constructed using the Agilent SureSelectXT Human All Exon V4 or V5. The libraries can be loaded onto a HiSeq Rapid v1 flowcell and sequenced using the Illumina HiSeq 2500 in rapid run mode with target read length of 2×100. Paired end sequencing data trimming of low quality bases and reads can be done using fastX_Toolkit. The reads that pass filtering can be aligned, using BWA-mem (Li 2009), to the most current human reference genome, HG19/GRCh37. Using SAMTOOLS, BWA output can be sorted, indexed and filtered for PCR duplicates. The resulting file can be locally realigned and target loci can be marked using GATK IndelRealigner and TargetIntervals. Non-repetitive regions can be mined for relevant SNPs and indels with SNPeffect. MSTs can be analyzed using our lab designed custom MST minor-allele software described in the next section.

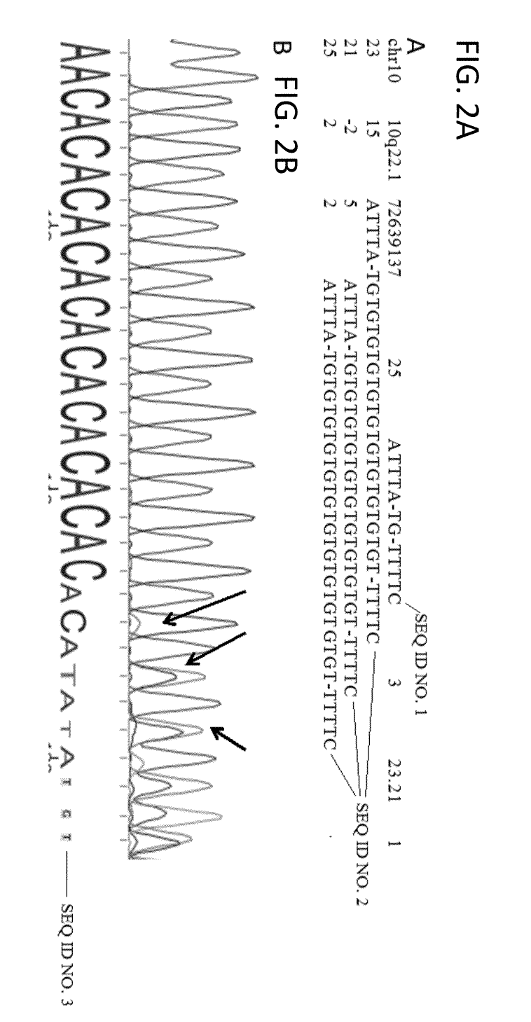

Microsatellite minor-allele custom software: Somatic MST variability can be evaluated using our custom MST minor-allele caller, an extension of our GenoTan and ReviSTER software (Tae H, Kim D Y, McCormick J, Settlage R E, Garner H R: Discretized Gaussian mixture for genotyping of microsatellite loci containing homopolymer runs. Bioinformatics 2014, 30(5):652-9; and Tae H, McMahon K W, Settlage R E, Bavarva J H, Garner H R: ReviSTER: an automated pipeline to revise misaligned reads to simple tandem repeats. Bioinformatics 2013, 29:1734-1741). The minor-allele caller pulls marked MSTs from bam files using SAMTOOLs based on user defined flanking sequence length and predicted alignments. Reads with mapping quality scores below 10% or those with low base calling scores for nucleotides within the repeats are removed. Alleles are called only when a user defined number of confirming reads, with identical sequences in both directions of a paired-end run are identified. The final number of alleles is computed based on a user specified minimal requirement of substantiating reads. Loci used for analysis are determined by total depth, also a user defined parameter (see FIGS. 2A and 2B for output and Sanger confirmation).

Although MSTs are considered mutational “hot-spots” with a higher rate of somatic variability than non-repetitive DNA they are still relatively stable with only a small percent of loci showing the presence of minor alleles (Zalman Vaksman N F, Hongseok Tae and Harold R Garner: Investigation of somatic dynamics of microsatellites in normal, FANCD2 and BRCA2 deficient cell lines with exome sequencing. Genome Research Submitted. Gemayel R, Vinces M D, Legendre M, Verstrepen K J: Variable tandem repeats accelerate evolution of coding and regulatory sequences. Annu Rev Genet 2010, 44:445-477; Ananda G, Walsh E, Jacob K D, Krasilnikova M, Eckert K A, Chiaromonte F, Makova K D: Distinct mutational behaviors differentiate short tandem repeats from microsatellites in the human genome. Genome Biol Evol 2013, 5:606-620; and Natalie C Fonville L J M, Zalman Vaksman, Harold R Garner: Microsatellites in the exome are predominantly single-allelic and invariant. Genome Biology Submitted).

This is most likely due to the multiple redundant mechanisms cells have evolved to suppress chromosomal instability and somatic variability. Because somatic mutation rates are under tight cellular control our observation is that SMV in “normal” healthy individuals is consistent with regards to mutation rates, types, and susceptible motifs. To that end the exome of unaffected siblings or patents of the DRDDs patients can be sequenced and determined; the rates of somatic variability based on motif sequence, MST length, proximity to coding regions and SNP and INDEL (expansion/contraction) bias, the differences in somatic variability between proximal MSTs (less than 10 bases apart) and non-proximal MSTs.

Exome or whole genome data from affected and related “unaffected” healthy individuals can be obtained. By sequencing available well characterized DRDD samples so as to reliably assess the spectrum of SMV signatures and their potential mechanistic connections, signatures can be obtained.

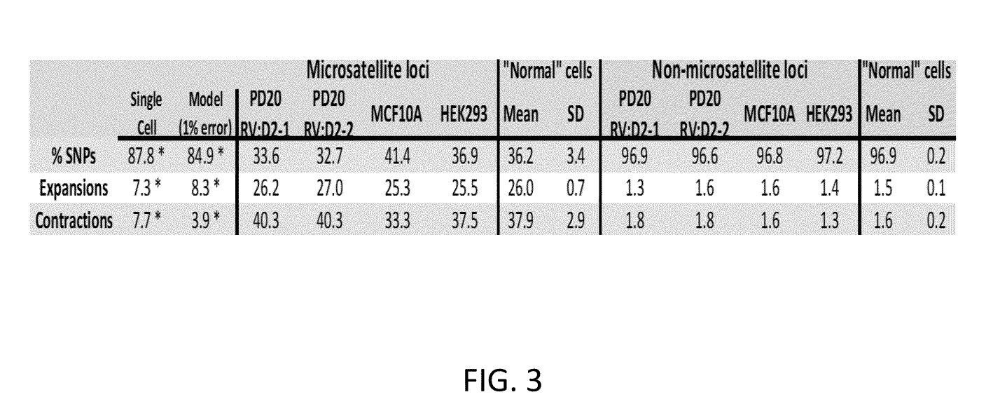

Accumulation of mutations can lead to catastrophic results for the organism therefore human cells have evolved multiple mechanics to maintain a low somatic mutation rate, even in MSTs (Natalie C Fonville L J M, Zalman Vaksman, Harold R Garner: Microsatellites in the exome are predominantly single-allelic and invariant. Genome Biology Submitted; Best B P: Nuclear DNA damage as a direct cause of aging. Rejuvenation Res 2009, 12:199-208; Hasty P, Campisi J, Hoeijmakers J, van Steeg H, Vijg J: Aging and genome maintenance: lessons from the mouse? Science 2003, 299:1355-1359; Williams L E, Wernegreen J J: Sequence context of indel mutations and their effect on protein evolution in a bacterial endosymbiont. Genome Biol Evol 2013, 5:599-605; and Denver D R, Morris K, Kewalramani A, Harris K E, Chow A, Estes S, Lynch M, Thomas W K: Abundance, distribution, and mutation rates of homopolymeric nucleotide runs in the genome of Caenorhabditis elegans. J Mol Evol 2004, 58:584-595). First, the inventors establish a “normal” pattern of SMV; they anticipate that the various SMV parameters must be fairly consistent between healthy unaffected subjects. Data using three commonly used “normal” cell lines, suggests that it is possible to establish a “normal” baseline for later comparison (Zalman Vaksman N F, Hongseok Tae and Harold R Garner: Investigation of somatic dynamics of microsatellites in normal, FANCD2 and BRCA2 deficient cell lines with exome sequencing. Genome Research Submitted). A within and between cell line SMV comparison was done using high coverage (80-120×) exome sequencing data obtained from PD20 RV:D2 (FANCD2 cells that were retrovirally corrected), MCF10A (breast epithelial cells) and HEK293 (embryonic kidney cells). An analysis of the mutation types in MSTs revealed nearly a 3:1 indel bias, which is in contrast to non-repetitive DNA, showing nearly a 50:1 SNP bias (the Table in FIG. 3). To confirm that these results were not a biased by on-chip amplification or sequencing error the inventors obtained an exome dataset from a study in which the authors isolated DNA from a single cell, PCR amplified it (20 cycles) and sequenced it (Hou Y, Song L, Zhu P, Zhang B, Tao Y, Xu X, Li F, Wu K, Liang J, Shao D, et al: Single-cell exome sequencing and monoclonal evolution of a JAK2-negative myeloproliferative neoplasm. Cell 2012, 148:873-885). For this dataset variability is predominantly a product of PCR error. An examination of the mutation bias in MST for single cell exome shows that ˜85% of the mutations are SNPs (the Table in FIG. 3). This result is replicated when an in-silico dataset with a 1% error rate is generated, which is similar to that is estimated by Illumina for the HiSeq. Further, the inventors found uniformity in the number of homozygotic and heterozygotic MST loci as well as the number of loci with minor alleles (the Table in FIG. 3).

Based on these results the inventors would anticipate that a more exhaustive analysis, one that can include SMV based on motif length, MST length, MST motif units can also remain consistent in healthy individuals. As the sample numbers increase the inventors anticipate the appearance of outliers due to the fact that many of the individuals that can be sequenced are the parents or siblings of the probands used in the other aims.

Establish changes in the pattern of somatic variability in patients and cell lines with DNA stability disorders leading to predisposition to cancer. DNA repair is essential for genomic stability and cell survival. In order to maintain genomic stability cells evolved multiple mechanisms to repair various types of DNA damage. DRDDs are congenital disorders that have impairments in one or more of these mechanisms, some of which are listed in the Table in FIG. 1 To date, only MMR has been implicated in MSI however very few other disorders have been tested for MSI. Since information on MST stability is clinically relevant the data compiled from this aim could assist clinicians in the treatment cancers for these patients. This is accomplished by determining the pattern of SMV in DRDD with impairments in various pathways. A disruption of any pathway leads to a distinct pattern of SMV and that the pathway disrupted can be predicted in unknown samples.

Samples from each disorder are exome sequenced and the data can be combined with sequencing results from other DNA repair disorders. Samples include those with Fanconi anemia, Bloom, Werner and Ruthmond-Thumson syndrome patients. For disorders such as Fanconi anemia and Xeroderma Pigmentosum (XP), which have multiple subgroups. For Fanconi anemia the subgroups analyzed include FANCA, C, D2 and G. For Cockayne syndrome, mutations in CSA (ERCC6) and XP.

DNA can be damaged in numerous ways including double strand breaks, inter and intrastrand cross-linking, nucleotide oxidation, deamination or insertion of inappropriate nucleotides. Cells have evolved specific machinery to remove and correct each type of damage (Sinha S, Singh R K, Alam N, Roy A, Roychoudhury S, Panda C K: Alterations in candidate genes PHF2, FANCC, PTCH1 and XPA at chromosomal 9q22.3 region: pathological significance in early- and late-onset breast carcinoma. Mol Cancer 2008, 7:84; and Hou Y, Song L, Zhu P, Zhang B, Tao Y, Xu X, Li F, Wu K, Liang J, Shao D, et al: Single-cell exome sequencing and monoclonal evolution of a JAK2-negative myeloproliferative neoplasm. Cell 2012, 148:873-885). DNA repair are grouped into two major classifications, SSDR and DSDR. For DSDR, subgroups include HR, NHEJ, single-strand annealing and cross-link repair. The major subgroups of SSR are BER, NER and MMR. DRDDs are disorders for which one or more of these pathways are disrupted. Individuals with these disorders accumulate somatic mutations at a much higher rate than a healthy individual leading to progeria like symptoms and a cancer predisposing. The types of mutations normally anticipated from each pathway are fairly defined and are based on the function of the protein conglomerate when attached to the DNA. For example, for Ruthmond-Thumson and Fanconi Anemia D1 patients are losses of large sections of the genome due to the inability to the shift to NHEJ after nucleotide rescission. However, for Bloom and Werner syndromes chromosomal instability is common due to the lack of Holiday junction resolution. In the case of SSDR the second strand is normally used as a template and therefore SNPs or short indels would be anticipated.

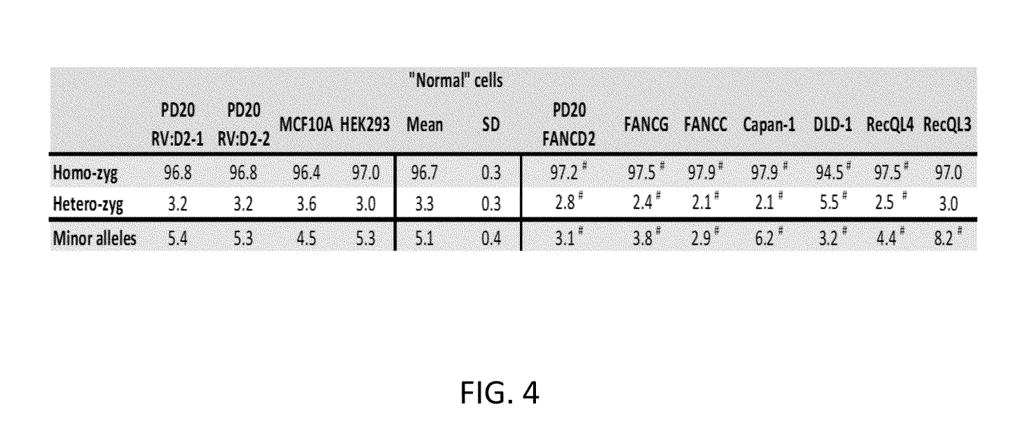

The only DNA repair pathways that has been directly associated to MSI is MMR. The inventors have sequenced and analyzed MSTs stability in a DLD-1, colorectal cancer derived cell line with defective MLH1 pathway (Glaab W E, Tindall K R, Skopek T R: Specificity of mutations induced by methyl methanesulfonate in mismatch repair-deficient human cancer cell lines. Mutat Res 1999, 427:67-78; Dexter D L, Spremulli E N, Fligiel Z, Barbosa J A, Vogel R, VanVoorhees A, Calabresi P: Heterogeneity of cancer cells from a single human colon carcinoma. Am J Med 1981, 71:949-956; and Russo M T, Blasi M F, Chiera F, Fortini P, Degan P, Macpherson P, Furuichi M, Nakabeppu Y, Karran P, Aquilina G, Bignami M: The oxidized deoxynucleoside triphosphate pool is a significant contributor to genetic instability in mismatch repair-deficient cells. Mol Cell Biol 2004, 24:465-474). These cells are MST unstable and are primarily diploid for all chromosomes. Results show a significantly greater number of heterozygotic loci as compared to repair unimpaired (“normal”) cell lines. Surprisingly, the number of loci with minor alleles was significantly less than the “normal” cell lines (the Table in FIG. 4). Based on these results the inventors would anticipate that individuals with Lynch syndrome to follow a similar pattern of SMV. Recent reports suggest that impairment in NER function does lead to the expansion or contraction of MST, specifically tri-nucleotide motifs (Goula A V, Pearson C E, Della Maria J, Trottier Y, Tomkinson A E, Wilson D M, 3rd, Merienne K: The nucleotide sequence, DNA damage location, and protein stoichiometry influence the base excision repair outcome at CAG/CTG repeats. Biochemistry 2012, 51:3919-3932; and Liu Y, Wilson S H: DNA base excision repair: a mechanism of trinucleotide repeat expansion. Trends Biochem Sci 2012, 37:162-172; and Goula A V, Berquist B R, Wilson D M, 3rd, Wheeler V C, Trottier Y, Merienne K: Stoichiometry of base excision repair proteins correlates with increased somatic CAG instability in striatum over cerebellum in Huntington's disease transgenic mice. PLoS Genet 2009, 5:e1000749) however, little is known about other motifs. Since both NER and BER are essential for repair of single strand damage the inventors would predict that impairments in either would result in increased mutation rate.

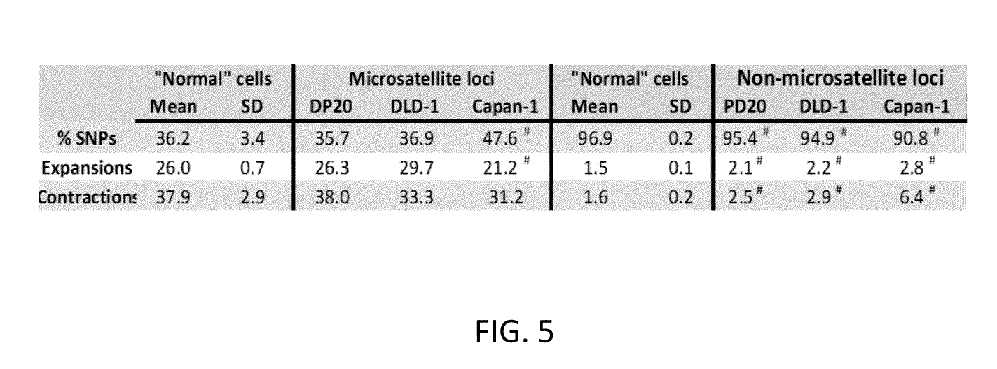

Damage resulting in double strand breaks requires a different approach to repair. In order to make an exact duplicate of the damaged DNA the cell requires a second, identical strand and a mechanism of filling in the damaged region, H R and impairments leads to LOH or loss of chromosomal regions. BRCA2 is an essential component of HR. Capan-1 cells are the only known cell line to have a completely non-functional BRCA2. An exome analysis of these cells showed that as anticipated, a significant loss of heterozygosity. Further, the inventors also detected a significant increase, 6.2% as compared to 5.1% (Capan-1 cells and “normal” cell line mean respectively) in the percentage of loci with minor alleles (the Table in FIG. 4). The inventors also found a difference between the SNP to indel, nearly a 1:1 ratio, for Capan-1 cells (the Table in FIG. 5).

FANC pathway is comprised of >14 genes responsible for interstrand cross-link repair (Pickering A, Zhang J, Panneerselvam J, Fei P: Advances in the understanding of Fanconi anemia tumor suppressor pathway. Cancer Biol Ther 2013, 14) and is associated with HR with a subgroup of the disorder being the BRCA2 gene, FANCD1. The severity of the disorder ranges greatly and is based on the subgroups. An important aspect of this aim is to determine if there is a correlation between severity and changes in SMV. Data for Fanconi anemia was obtained by sequencing the exomes of a FANCD2 cell line (PD20) and two patient samples, FANCC and FANCG. The data show that a significant LOH, as compared to controls (3.3, 2.8, 2.4, and 2.1% “normal” mean, FANCD2, C and G respectively). Although FANCD2 also showed a reduction in the number of heterozygotic loci it was much less then FANCC and G samples. The inventors found that the propensity for loci acquiring minor alleles was similarly reduced from “normal”, for all 3 samples.

Based on these results, it is possible to establish an SMV signature associated with specific disorders. The specific patterns of haplotypes distribution, minor alleles and mutational bias (SNP vs indel) appear to be pathway specific. Further, although trends remain the same slight differences have emerged when different genes are affected within the same disorder. This is seen in the slight difference between FANCD2, and FANCC/G. These results demonstrate that comparisons of SMV can be used in a more predictive manner based on signature.

### Example 2

Methods

Cells, DNA prep and sequencing: HEK (human embryonic kidney) and MCF10A (immortalized breast epithelial) and HEK293 (human embryonic kidney) cells were obtained from ATCC. PD20 and PD20 RV:D2 (FANCD2 and FANCD2 retrovirally corrected) cell lines were obtained from the Fanconi Anemia Foundation (Eugene Oreg.). Sequencing data for Capan-1 cells was previously published by Barber and coworkers (Barber L J, Rosa Rosa J M, Kozarewa I, Fenwick K, Assiotis I, et al. (2011) Comprehensive genomic analysis of a BRCA2 deficient human pancreatic cancer. PLoS One 6: e21639). PD20, PD20 RV:D2 and HEK293 cells were grown at 37° C. with 5% CO2, in DMEM supplemented with 10% FBS (Invitrogen) and 1× pen/strep (Invitrogen) to 80% confluence. MCF10A cells were grown to confluence in DMEM/F12 medium (Invitrogen, Carlsbad, Calif.), supplemented with 5% horse serum (Invitrogen), antibiotics—1× Pen/Strep (Invitrogen), 20 ng/mL EGF (Peprotech, Rocky Hill, N.J.), 0.5 mg/mL hydrocortisone (Sigma), 100 ng/mL cholera toxin (Sigma), and 10 μg/mL insulin (Sigma) at 37° C. with 5% CO2. All cell lines were collected by trypsinazation and prepared for DNA extraction. DNA was extracted using the Qiagen DNAeasy kit (Qiagen) as per manufacturer instructions.

Sequencing and analysis pipeline: Exome paired-end libraries were prepared using the Agilent (Chicago, Ill.) SureSelectXT Human All Exon V4 capture library. 2×100 bp reads were obtained using an Illumina (San Diego, Calif.) HiSeq 2500 instrument in Rapid Run mode on a HiSeq Rapid v1 flowcell. Indexed reads were de-multiplexed with CASAVA v1.8.2.

Paired-end sequencing reads were trimmed using fastX_Toolkit and aligned to HG19/GRCh37 human reference genome using BWA-mem. The output was then sorted, indexed and PCR duplicates were removed using SAMTOOLS (Li H, Handsaker B, Wysoker A, Fennell T, Ruan J, et al. (2009) The Sequence Alignment/Map format and SAMtools. Bioinformatics 25: 2078-2079). Bam files were then locally realigned and target loci marked using GATK IndelRealigner and TargetIntervals. MST alleles were retrieved and analyzed using software described in the next section.

Microsatellite minor-allele software: A catalogue of MST loci was generated from the HG19/GRCh37 reference genome using Tandem Repeats Finder (Benson G (1999) Tandem repeats finder: a program to analyze DNA sequences. Nucleic Acids Res 27: 573-580) (with the following parameters: 2.7.7.80.10.18.6). The list was filtered to remove any loci that were shorter than 8 nucleotides, had less than 3 copies of a given motif unit or were below 85% sequence purity. Duplicated loci were identified based on sequence purity and sequence length and were removed.

MSTs were analyzed using a custom MST minor-allele caller based on GenoTan and ReviSTER software (Tae H, Kim D Y, McCormick J, Settlage R E, Garner H R (2013) Discretized Gaussian mixture for genotyping of microsatellite loci containing homopolymer runs. Bioinformatics 2014 30(5):652-9; and Tae H, McMahon K W, Settlage R E, Bavarva J H, Garner H R (2013) ReviSTER: an automated pipeline to revise misaligned reads to simple tandem repeats. Bioinformatics 29: 1734-1741), which were developed by this group to improve MST haplotype predictions. The minor-allele caller extracts marked MSTs from bam files using SAMTOOLs. MST loci are called based on predicted alignments and an adjustable length flanking sequence (this study used either 5 or 7 nucleotide sequence). Reads with low base call scores (below a base score of 28) for nucleotides within the repeats and those with mapping quality score below 10% were eliminated. Alleles are initially called only when two or more reads, verified in both directions of a paired-end run, have the same sequence. All alleles for a given locus are binned with the number of supporting paired-end reads. The final number of alleles is computed based on a user specified minimal requirement of substantiating reads (for this study the minimum number of substantiating reads is either 2 or 3 reads per allele). If more than one allele per locus was found, zygosity and the sequence length difference from the most common allele were recorded. Heterozygotic loci were called using the following criteria as described and confirmed in the GenoTan and ReviSTER manuscripts (Tae H, Kim D Y, McCormick J, Settlage R E, Garner H R (2014) Discretized Gaussian mixture for genotyping of microsatellite loci containing homopolymer runs. Bioinformatics 30(5):652-9; and Tae H, McMahon K W, Settlage R E, Bavarva J H, Garner H R (2013) ReviSTER: an automated pipeline to revise misaligned reads to simple tandem repeats. Bioinformatics 29: 1734-1741) 1) it is the second most common allele, 2) The number of confirming reads is greater than 25% of the total reads for the locus or greater than 50% of the depth for the most common allele, if the total is below 25% of the total depth.

In addition to MST loci, the inventors also generated a somatic variability profile for non-MST loci. To make the data comparable the inventors randomly selected 3 million loci, each consisting of 15 nucleotides segments, from the HG19 genome. The inventors then filtered out any loci that intersected with our MST and were left with over 2 million loci. The same pipeline as for MSTs was used to generate the data for non-MST loci. This data yielded information on the number of loci with minor alleles and type of mutation (SNPs and INDELs).

Sequence validation and allele calls validated by independent Sanger sequencing method. The MST minor-allele caller the inventors use in this Example is a modified version of a published and experimentally verified code, however to further validate the multi-allele capability of the modified code 30 loci, including 17 showing multiple alleles, were verified using Sanger sequencing. Additional data (not shown) shows the data from the minor-allele caller output at one of these loci, chr10:72639137-72639161, at which the inventors would predict at least 3 alleles to be present in this sample (MCF10A) with lengths of 21, 23, and 25 nucleotides. Sanger sequencing confirmed that multiple alleles were present, with the alleles being greater than 21 nucleotides long. Of the 30 loci 28 loci verified the genotype and 14 of 17 loci with minor alleles also had visible minor alleles by Sanger sequencing.

Modeling error rates to establish rules that differentiate errors from high confidence minor alleles: Two methods were used to generate models of NGS runs for chromosomes 17 and 21; 1) WgSim, a commonly used paired-end read generator and 2) in-house designed generator. Both methods were set to have a per nucleotide error rate between 0.5% and 5%. The major difference between the two methods was that wgsim was used to obtain modeling data with fairly similar coverage (read depth) across the reference chromosome while the lab-designed algorithm allowed for a more variable coverage as is observed in a typical next-generation sequencing run. The generated fastq files were run through the same pipeline as actual real sequencing data. The accuracy of the pipeline was analyzed by the verification of the predicted alignment. Predicted error rates ranged between 1.3% and 1.9%, with the majority of errors due to misalignments.

Results

The inventors modified a previously published and verified MST genotyper (Tae H, Kim D Y, McCormick J, Settlage R E, Garner H R (2014) Discretized Gaussian mixture for genotyping of microsatellite loci containing homopolymer runs. Bioinformatics 30(5):652-9) to enumerate all possible alleles present within next-generation data, as opposed to only capturing the most common (haplotype) alleles. The inventors first characterized the error which may cause false positive allele calls via a parametric sensitivity study conducted on in-silico generated data, and showed that their measure can then be used to accurately quantify minor alleles and thus be used to distinguish between mutational mechanisms that are exhibited in different cell lines. To accomplish this, the inventors establish a baseline SMV profile from DNA repair proficient cell lines, and compared this to what is seen in cell lines with various DNA repair defects.

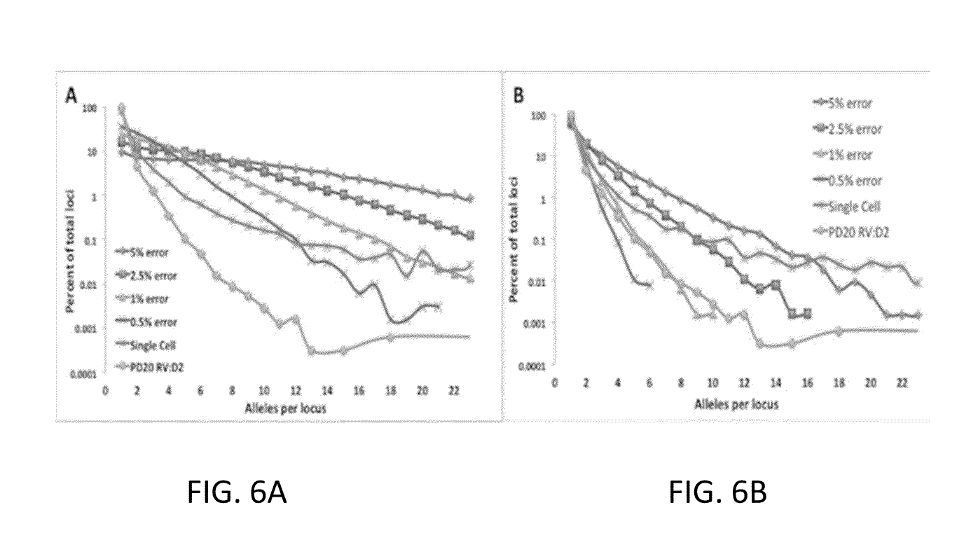

Characterizing the effect of sequencing error on minority allele calling: This analysis evaluates each MST locus to establish the one or two alleles that define the genotype, then it robustly calls additional non-haplotype or ‘minor’ alleles that are present at lower frequency within next-generation data. However, the accuracy of such minority allele calls can be significantly affected by sequencing errors found within the raw reads that map to each locus. To minimize the number of false positive ‘alleles’, the inventors first established the minimal number of reads necessary for confirming an allele in the presence of typical next-generation errors. It has been established by a number of studies that 3 reads mapped to a loci is sufficient to properly call major alleles (McIver L J, McCormick J F, Martin A, Fondon J W, 3rd, Garner H R (2013) Population-scale analysis of human microsatellites reveals novel sources of exonic variation. Gene 516: 328-334; Lauren J McIver N C F, Enusha Karunasena, Harold R Garner (Submitted) Microsatellite genotyping reveals a signature in breast cancer exomes. Breast Cancer Research and Treatment; and Natalie C Fonville L J M, Zalman Vaksman, Harold R Garner (Submitted) Microsatellites in the exome are predominantly single-allelic and invariant. Genome Biology). To corroborate this, the inventors created an in-silico sequencing data set for chromosomes 21 and 17, with randomly generated errors ranging from 0.5% to 5% which mimicked next-generation sequencing data in both the error types that were created and read coverage per locus (results depicted in FIGS. 6A and 6B).

The inventors first determined the parameters required to optimize the measurement of the fraction of loci without minor alleles in sequencing data with the above-mentioned error rates. The sequencing data generator produced between 8 and 10.5 million reads that contained over 58,000 targeted MSTs. Over 98.5% of the reads mapped correctly with an accuracy of over 99.8% in coding regions (regions captured by exome sequencing). The accuracy of zygosity calls was over 99.98% for all error rates. Next the inventors varied the minimum number of reads covering a locus required to call an allele. Changing the threshold from 2 confirming reads (FIG. 6A) to 3 confirming reads (FIG. 6B) statistically and significantly decreased the fraction of loci with more alleles than the haplotype number (1 if homozygotic or 2 if heterozygotic). Using a threshold of 2 confirming reads per allele, the fraction of loci without minor alleles identified (due to sequencing errors being interpreted as alleles) was 19-62% for simulated data sets with error rates ranging between 5%-0.5% respectively (FIG. 6A), indicating that requiring only 2 reads to identify an allele leads to a high level of false alleles. By increasing the threshold to 3 confirming reads the percent of loci without minor alleles increases to 73-99% for the same data set (FIG. 6B). By increasing to 4 confirming reads per allele the inventors further increase the number of loci without minor alleles 87%-99%. However, at error rates close to the actual HiSeq rates (of ˜1%), the inventors only saw a modest increase in the number of loci without minor alleles, a change from 97% (3 reads per allele) to 99% (4 reads per allele). This is in contrast to an increase from 61% with 2 reads per allele to 97% with 3 confirming reads per allele.

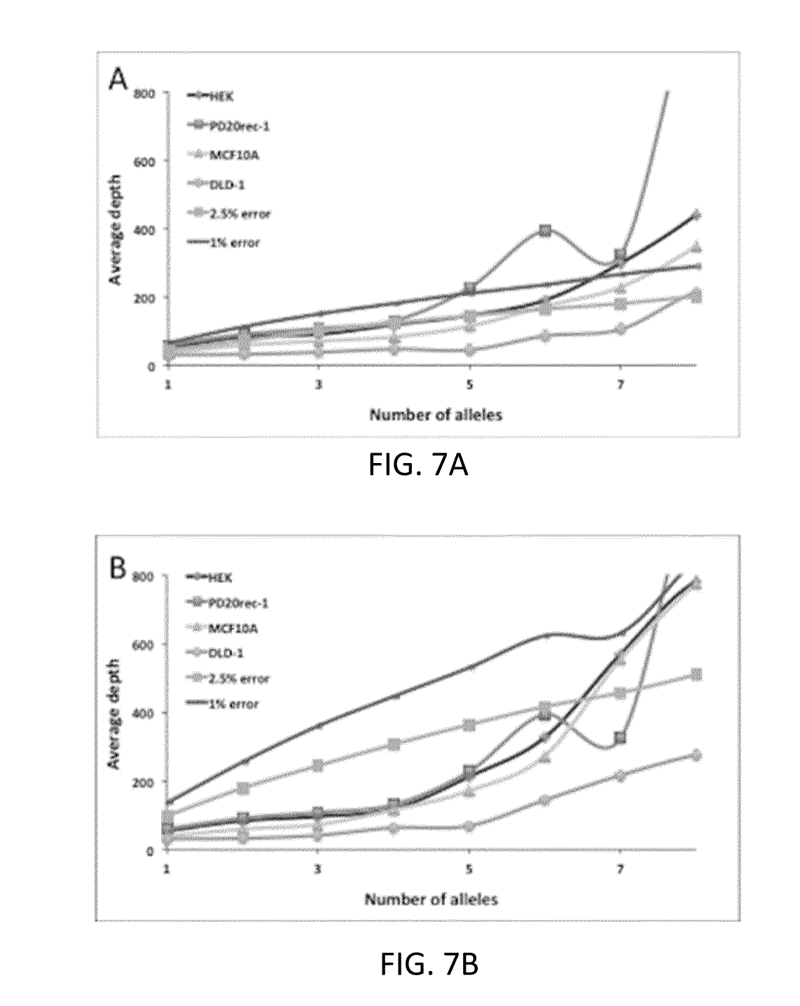

The inventors next examined how sequencing error might affect the number of alleles present in their data. To do this the inventors used modeling data with error rates similar to the actual HiSeq error rate (1%) and 2.5% error (FIGS. 7A and 7B), and determined the average read depth per locus with increasing alleles. For the in-silico generated data, the inventors found a linear increase in the total read depth as the number of alleles increased (using 2-4 confirming reads per allele) up to 8 alleles (FIGS. 7A and 7B). A comparison of these results to actual sequencing data from our cell lines (discussed in more detail later) shows that when 3 or more reads are required to confirm an allele, the number of alleles called for a given read depth is greater than what would be expected from error, even at a rate of 2.5% which is substantially more than the observed next-generation error rate of 1% (FIG. 7B), i.e. more alleles are called at a lower read depth in the actual data than would be present due to error. Based on these results, requiring a minimum of 3 reads covering a locus to confirm an allele minimizes the number of ‘false’ alleles being identified due to sequencing error.

Polymerase slippage vs. nucleotide misincorporation: Another potential source of error in calling alleles from sequencing data is amplification errors induced during the library preparation process (Schmitt M W, Kennedy S R, Salk J J, Fox E J, Hiatt J B, et al. (2012) Detection of ultra-rare mutations by next-generation sequencing. Proc Natl Acad Sci USA 109: 14508-14513). These errors would likely be present at higher frequency than errors generated during sequencing (Gundry M, Vijg J (2012) Direct mutation analysis by high-throughput sequencing: from germline to low-abundant, somatic variants. Mutat Res 729: 1-15), and therefore cannot be minimized by solely increasing the minimum read coverage (as above). Somatic mutation of MSTs is primarily associated with polymerase slippage, which is thought to cause the characteristic INDEL bias (Schmitt M W, Kennedy S R, Salk J J, Fox E J, Hiatt J B, et al. (2012) Detection of ultra-rare mutations by next-generation sequencing. Proc Natl Acad Sci USA 109: 14508-14513; Kanagawa T (2003) Bias and artifacts in multitemplate polymerase chain reactions (PCR). J Biosci Bioeng 96: 317-323; and Meyerhans A, Vartanian J P, Wain-Hobson S (1990) DNA recombination during PCR. Nucleic Acids Res 18: 1687-1691). In contrast, nucleotide misincorporation errors during in-vitro amplification would be predicted to lead primarily to SNPs in sequencing data (Brodin J, Mild M, Hedskog C, Sherwood E, Leitner T, et al. (2013) PCR-induced transitions are the major source of error in cleaned ultra-deep pyrosequencing data. PLoS One 8: e70388). Both of the mentioned DNA synthesis methods would lead to an increase in the number of loci with non-haplotype alleles, however with a predicted variation pattern that is distinctly different. To differentiate between the two predicted SMV patterns including minority alleles, and to assess the influence of nucleotide mis-incorporation/amplification error on our results, the inventors compared a standard exome sequence from cells which are proficient for DNA repair (described later) that did not undergo whole genome amplification (WGA) with data from the sequencing of a single cell (Hou Y, Song L, Zhu P, Zhang B, Tao Y, et al. (2012) Single-cell exome sequencing and monoclonal evolution of a JAK2-negative myeloproliferative neoplasm. Cell 148: 873-885) which would be expected to have no somatic variation within the sample, but has necessarily undergone WGA to generate the quantity of DNA necessary for sequencing. Therefore, for the WGA sample, presumably all non-haplotype alleles present are due to amplification error. As expected, genome amplification increases the number of loci with non-haplotype alleles (FIGS. 6A and 6B) to 11.3% and 7% of the total with a threshold of 2 and 3 reads, respectively. The DNA repair proficient cells, which did not undergo extensive amplification, were only decreased by 1.7%, from 7% to 5.3%, by altering the minimum read cutoff. From this it can be concluded that neither errors during library prep nor during the sequencing run account for more than 4 percent of the total non-haplotype alleles detected.

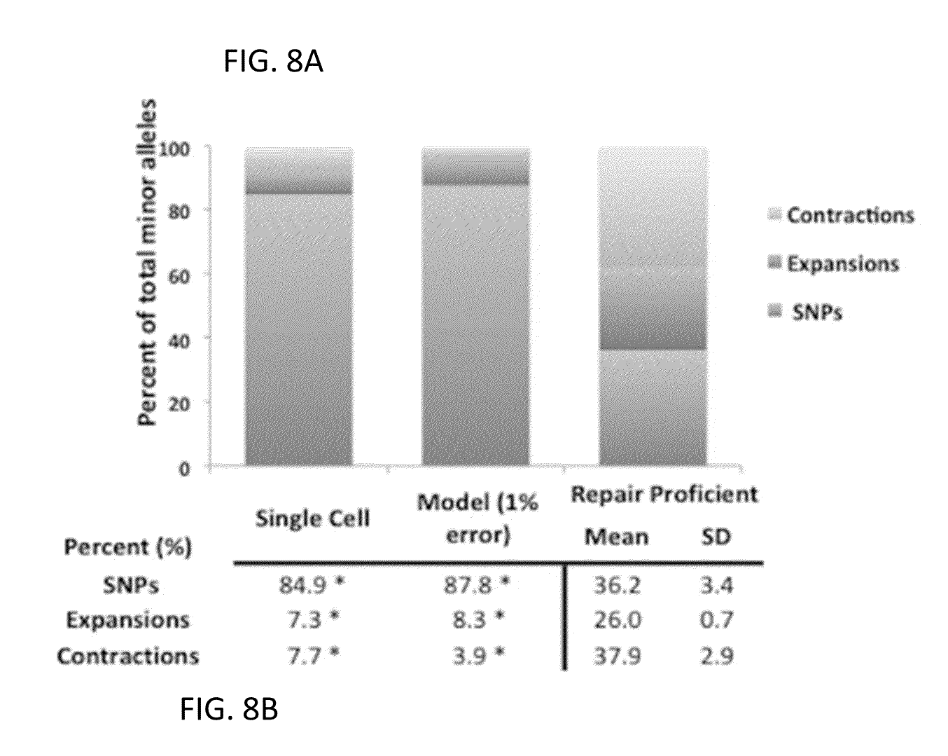

Approximately 85% of mutations found within microsatellite loci in the WGA single-cell data were SNPs, which is expected as a consequence of polymerase errors during amplification. These results were comparable to those predicted by our model, which showed that ˜88% of the total minor alleles were composed of alleles carrying SNPs rather than INDELs (FIGS. 8A and 8B). In contrast, SNPs account for only 36% (±3.4%) of the total minor alleles in DNA repair proficient cell lines. In addition, although for all the DNA repair proficient cell lines the most common MST motifs with minor alleles observed were mono-nucleotide repeats found within 56%-66% of loci, loci containing tri-nucleotide motifs accounted for over 55% of the total loci with minor alleles in the WGA data. These results further support the hypothesis that this approach can differentiate between distinct MST mutational profiles: INDELs, particularly at mono-nucleotide runs predominantly reflect DNA repair proficient biological SMV whereas SNPs in MSTs, particularly at tri-nucleotide motif containing loci are predominantly amplification-induced errors or potentially due to altered DNA maintenance capacity. This is further supported by a similar study that has found that the majority of MSTs that are variable within the normal population (individuals sequenced as part of the 1,000 Genomes Project) are predominantly INDELs at mono-nucleotide runs (Natalie C Fonville L J M, Zalman Vaksman, Harold R Garner. Microsatellites in the exome are predominantly single-allelic and invariant. Genome Biology (Submitted)).

MST vs non-MST regions: MSTs are considered to be more susceptible to mutations than the surrounding non-repetitive DNA regions (Bagshaw A T, Pitt J P, Gemmell N J. (2008) High frequency of microsatellites in S. cerevisiae meiotic recombination hotspots. BMC Genomics 9: 49; Yoon K, Lee S, Han T S, Moon S Y, Yun S M, et al. (2013) Comprehensive genome- and transcriptome-wide analyses of mutations associated with microsatellite instability in Korean gastric cancers. Genome Res 23: 1109-1117; and Mestrovic N, Castagnone-Sereno P, Plohl M (2006) Interplay of selective pressure and stochastic events directs evolution of the MEL172 satellite DNA library in root-knot nematodes. Mol Biol Evol 23: 2316-2325).

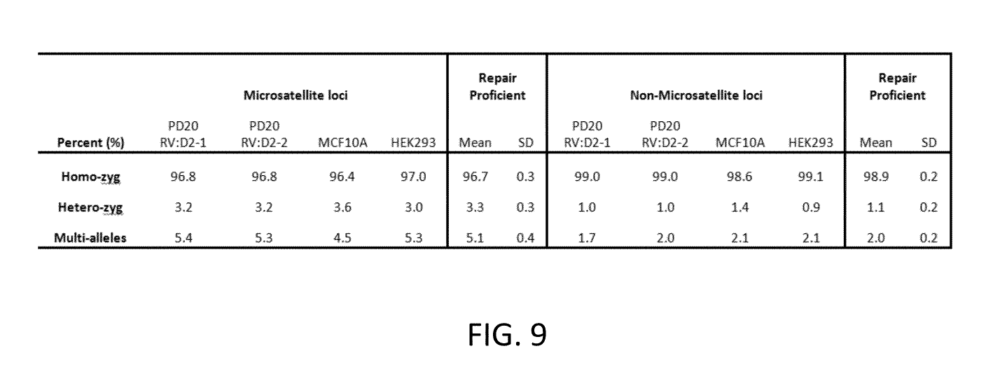

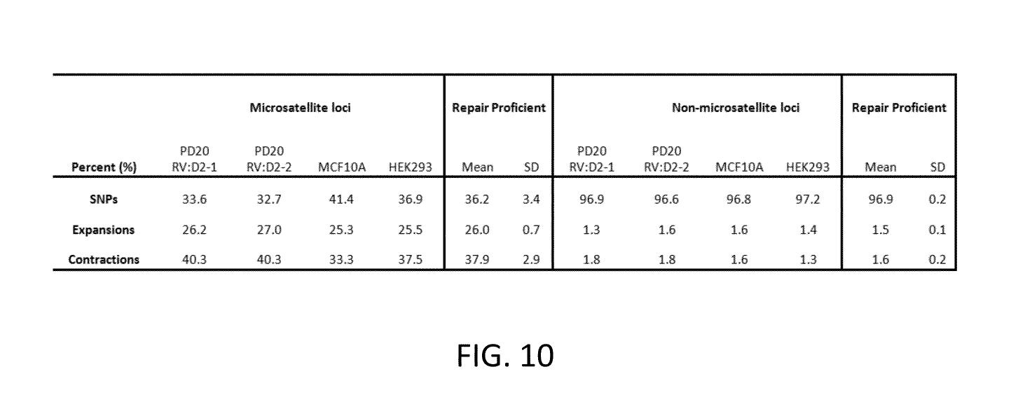

Because of this, one could expect that non-MST regions would have less somatic variability (non-MST equivalent of SMV) than MST regions. In order to perform a fair comparison with the MST data, 2 million segments consisting of 15 nucleotides each were randomly selected throughout the genome. The same analysis as was performed on loci containing MSTs was also applied to these non-MST regions. It was found that for these non-MST loci the average fraction of loci that were homozygotic was 98.9% with a standard deviation of 0.2, while only 96.7% of the MST containing loci was homozygotic. Even more significant, only 2% (standard deviation of 0.2) of the non-MST loci (homozygotic and heterozygotic) had minor alleles, while 5.1% of the MST loci harbored minor alleles (the Table in FIG. 9). Further, a comparison of SNP and INDEL distributions indicated that, unlike MST regions where INDEL variations prevail (64%), SNPs account for the majority (96.9%) of the differences in minor alleles at non-MST loci (the Table in FIG. 10). Taken together, these results confirm that, consistent with the literature, MSTs are more susceptible to mutation (Fonville N C, Ward R M, Mittelman D (2011) Stress-induced modulators of repeat instability and genome evolution. J Mol Microbiol Biotechnol 21: 36-44; Bagshaw A T, Pitt J P, Gemmell N J (2008) High frequency of microsatellites in S. cerevisiae meiotic recombination hotspots. BMC Genomics 9: 49; and Payseur B A, Jing P, Haasl R J (2011) A genomic portrait of human microsatellite variation. Mol Biol Evol 28: 303-312; Jarne P, Lagoda P J (1996) Microsatellites, from molecules to populations and back. Trends Ecol Evol 11: 424-429).

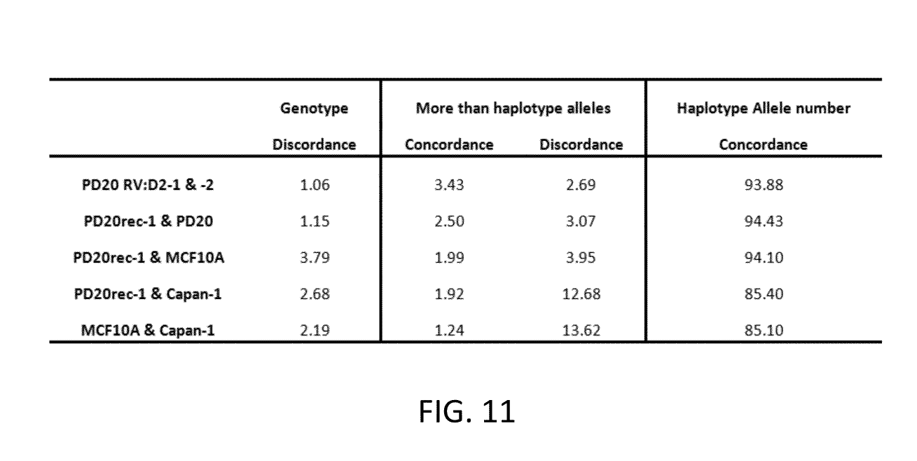

Reproducibility within a cell line: The objective of this study is to characterize the pattern of SMV from DNA repair proficient cells and then compare to cell populations in which DNA repair is compromised. SMV changes associated with disease can likely be subtle and require highly reproducible control data. To test the reproducibility of SMV measurements within a cell line, two biological replicate cultures of PD20 RV:D2 (PD20 RV:D2-1 and PD20 RV:D2-2) cells were grown separately and sequenced. PD20 RV:D2 are fibroblasts derived from an individual with Fanconi Anemia subgroup D2, retrovirally complimented with a functional copy of FANCD2 (Ohashi A, Zdzienicka M Z, Chen J, Couch F J (2005) Fanconi anemia complementation group D2 (FANCD2) functions independently of BRCA2- and RAD51-associated homologous recombination in response to DNA damage. J Biol Chem 280: 14877-14883). Using a minimum read depth cutoff of 15 to genotype a given loci, the inventors successfully called over 280K and 250K loci (at an average depth of 52 and 45 reads per locus) for PD20 RV:D2-1 and 2 respectively. Both samples showed a similar SNP to INDEL ratio, with INDELs making up over ˜67% of the minor alleles (the Table in FIG. 10). A genotype analysis showed that approximately 96.8% of called loci were homozygous while heterozygosity was observed in ˜3.2% of the loci called (the Table in FIG. 9). Comparison of those loci that were called in both samples shows that haplotype discordance (i.e. homo- or heterozygotic using standard genotyping) was 1.1% (the Table in FIG. 11), of which 92% were due the fraction of reads supporting a second allele being below the haplotype threshold (see Method) and was therefore counted as a minor allele instead of a second haplotype allele, as is the convention in established genotype callers. Only 173 discordant loci were due to sequence differences between the two samples.

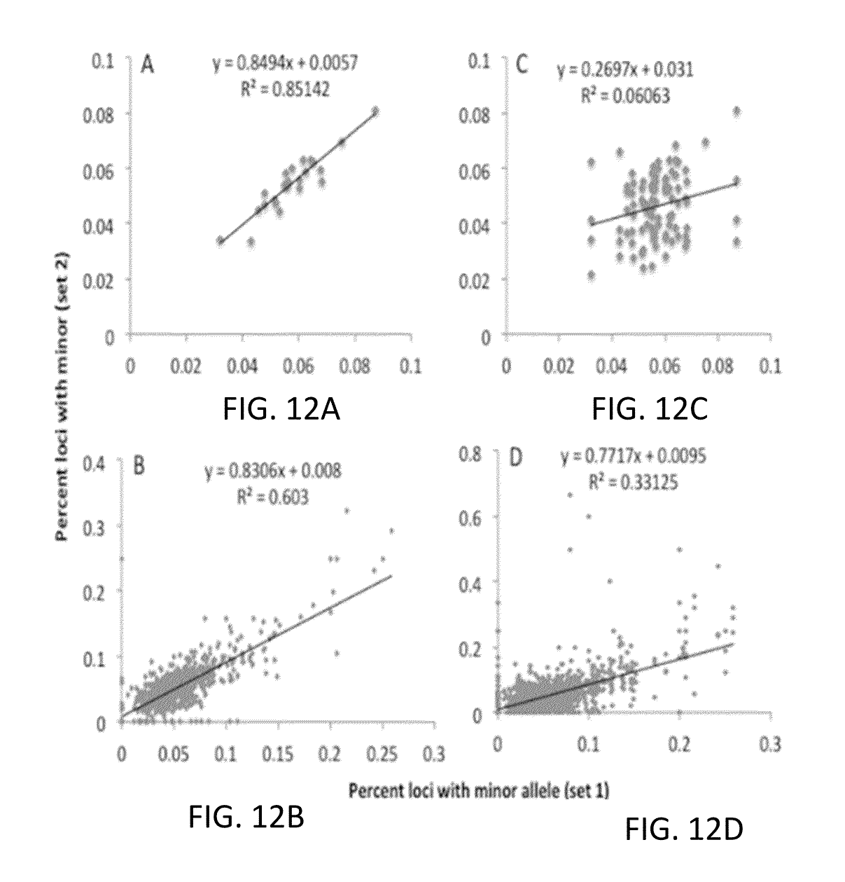

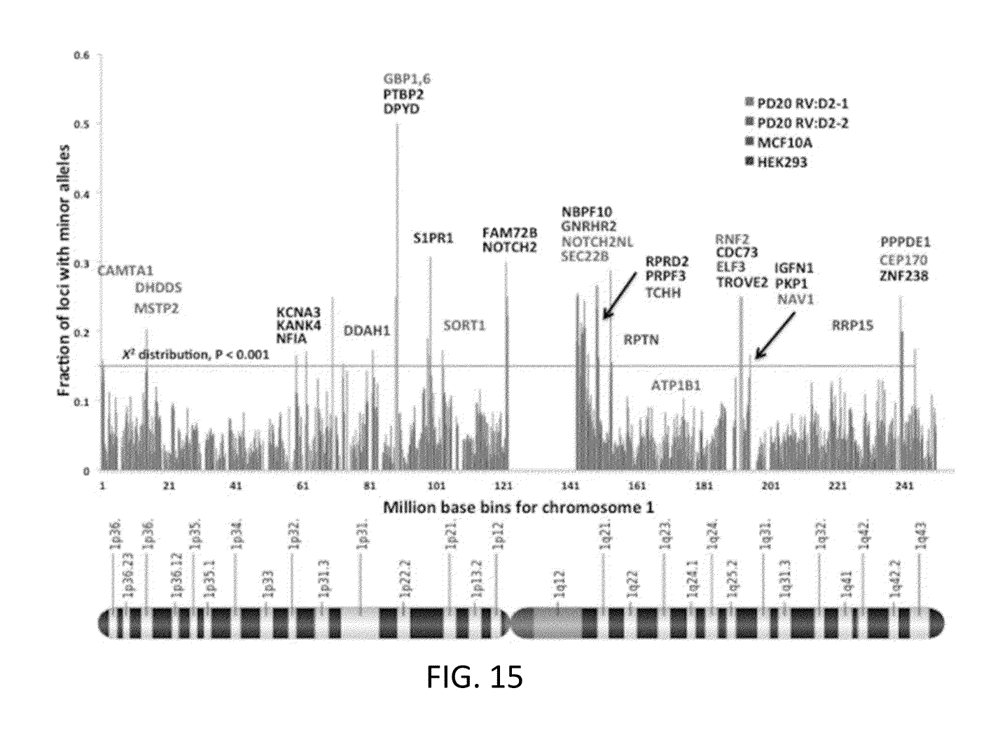

For the purpose of this study SMV is defined by the presence of variant MST alleles that are supported by a minimum of 3 confirming reads but do not contribute to haplotype. An analysis of variant MST alleles found a total of 5.4% and 5.3% of MST loci in the PD20 RV:D2-1 and 2 samples, respectively, had 1 or more minor alleles (the Table in FIG. 9). The concordance of loci without minor alleles in either sample is 93.9% while 3.4% of loci have at least one minor allele in both samples. Concordance according to this specification means a locus has minor alleles or the same haplotype in multiple samples. Conversely, discordance, where a locus in only one of the compared samples had minor alleles, was 2.7% (the Table in FIG. 11). To confirm the significance of these values, the inventors calculated the probabilities of concordance and discordance based on a cohort of randomly selected loci (5.4% and 5.3% of a total samples), which was <0.25% concordant, and compared with our results. Using a Pearson's goodness of fit X², the inventors verified that the concordant loci are not randomly distributed (p<0.0001). To determine within cell line reproducibility the inventors compared the percent of loci having minor alleles by chromosome as a whole and binned into a million base regions. A linear regression model comparing the percent of loci with minor alleles for each chromosome shows a significant correlation (R²=0.85 and p<0.001) between two independently cultured samples (FIG. 12A). Similarly, a comparison of the binned chromosome also shows a significant correlation (R²=0.60 and p<0.001, FIG. 12B). Visualization of the distribution of fraction of MST loci showing somatic variation in a representative chromosome (chr1), depicted in FIG. 15, indicates specific chromosomal regions that may harbor SMV “hot-spots”. An evaluation of MST loci in translated (exon) regions found over 820 genes containing MSTs with a minimum of 2 minor alleles in both PD20 RV:D2 samples, with some of genes found within segments of chromosome 1 with increased SMV depicted in FIG. 15.

Taken together these results support the inventors' hypothesis that this method truly reflects SMV rather than error generated during sequencing and that the results are highly reproducible. The data further suggests that within an individual or cell line, specific genomic regions may contain MSTs that are more susceptible to somatic variability.

Reproducibility between cell lines: To begin to establish a SMV baseline for DNA repair proficient cells, the inventors compared the haplotype, minor allele and SNP/INDEL distributions for two DNA repair proficient cell lines and the PD20 RV:D2 cells discussed above. MCF10A cells are immortalized breast epithelial cells derived from a healthy human female and HEK293 cells are a human embryonic kidney cell line derived from a healthy male fetus. Sequencing produced over 45 million reads with over 170K microsatellite loci called at an average depth of 42 reads per locus for HEK293 cells and over 190K microsatellite loci called at an average depth of 39 reads per locus for MCF10A cells. Considering major alleles only, 96.4% and 97.0% of all MST loci, respectively, are homozygotic (the Table in FIG. 9). The average fraction of loci with minor alleles for all three cell lines was 5.1% with a standard deviation of 0.4%. Although MCF10A cells had fewer loci with minor alleles than the PD20 RV:D2 and HEK293 cells (4.5% compared with 5.3% and 5.4% respectively, the Table in FIG. 9), and showed a difference in the fraction of secondary alleles with SNPs compare to INDELS (the Table in FIG. 10), MCF10A was not considered an outlier (using Grubb's test for outliers). When the inventors compared the haplotype and minor allele concordance between two non-related cell lines, MCF10A and PD20 RV:D2, they found that 3.8% of loci have different genotypes with only 60% due to haplotype differences. For those loci with minor alleles, discordance is 4.0% and concordance is only 2.0%, the result is significantly above what would be anticipated by chance with Pearson's X² (i.e. <0.3%). Interestingly, a full factorial comparison of the fraction of loci with minor alleles for each chromosome, using a linear regression model, found a non-significant correlation (R²=0.061 and p<0.23, FIG. 12C). However, a correlation using the 1 million base bins is significant with an R² value of 0.33 and a p<0.0001 (FIG. 12D), supporting the concept that certain regions contain minor allele susceptibility hot spots. These results demonstrate substantial reproducibility between unrelated independently grown DNA repair proficient cell lines even when the samples are derived from different tissues of origin. These results also suggest that a baseline profile of SMV can be established for DNA repair proficient cells to compare to cell lines with DNA repair defects.

SMV in cells with compromised DNA repair capacity: Thus far the inventors have established that (1) three DNA repair proficient cell lines show similar SMV with low variability both within and between cell lines and that (2) the inventors can differentiate between different SMV trends based on the ratio of INDELs to SNPs. However, the larger goal of this study is to compare SMV patterns between cell lines representative of healthy individuals and those that may have altered DNA repair capacity. To test this, the inventors evaluated 3 cell lines commonly used to study DNA repair and stability. DLD-1 cells are MST instability (MSI) high colon cancer cell line, impaired in Mismatch repair (MMR), selected as positive controls for this study (Chen T R, Hay R J, Macy M L (1983) Intercellular karyotypic similarity in near-diploid cell lines of human tumor origins. Cancer Genet Cytogenet 10: 351-362). Capan-1 cells were sequenced previously (Barber L J, Rosa Rosa J M, Kozarewa I, Fenwick K, Assiotis I, et al. (2011) Comprehensive genomic analysis of a BRCA2 deficient human pancreatic cancer. PLoS One 6: e21639) and are a BRCA2− cell line that can propagate in culture. PD20 cells are from a FANCD2(−) cell line from which the PD20 RV:D2 cells were derived (Ohashi A, Zdzienicka M Z, Chen J, Couch F J (2005) Fanconi anemia complementation group D2 (FANCD2) functions independently of BRCA2- and RAD51-associated homologous recombination in response to DNA damage. J Biol Chem 280: 14877-14883). Both the Capan-1 cells and the PD20 cells have mutations in genes that are involved in normal DNA repair (homologous recombination and interstrand cros slink repair, respectively).

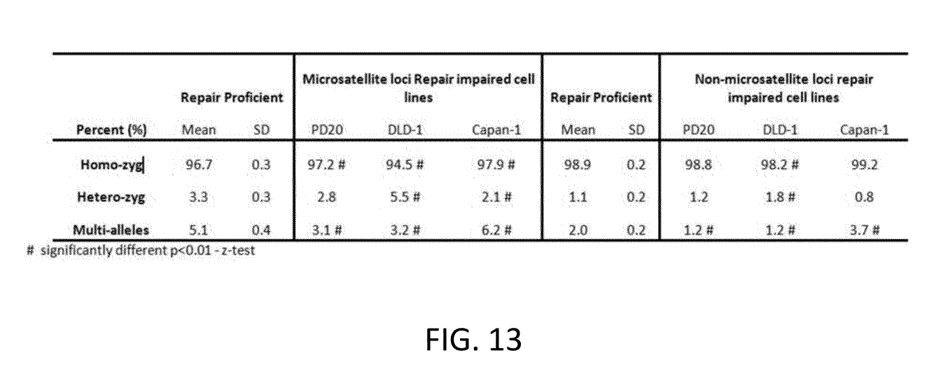

For DLD-1 and PD20 cells, the number of loci that passed filters ranged between 185K and 260K with an average depth of between of 56 and 62 reads per locus respectively. Only 124K loci were called for Capan-1 cells, with an average depth of 71 reads per locus. To capture MST differences between the DNA repair proficient and DNA repair defective cell lines the inventors first evaluated haplotypes and the presence of minor alleles for each cell line. Both DLD-1 and Capan-1 cells significantly differ with respect to haplotype distribution from DNA repair proficient cells (the Table in FIG. 13). Capan-1 cells showed a significant decrease in heterozygotic loci, 2.1% compare to 3.3% for DNA repair proficient, which was anticipated due to the known trend for loss of heterozygosity in these cells as reported in the literature due to gene conversion in the absence of BRCA2 (Holt J T, Toole W P, Patel V R, Hwang H, Brown E T (2008) Restoration of CAPAN-1 cells with functional BRCA2 provides insight into the DNA repair activity of individuals who are heterozygous for BRCA2 mutations. Cancer Genet Cytogenet 186: 85-94; and Butz J, Wickstrom E, Edwards J (2003) Characterization of mutations and loss of heterozygosity of p53 and K-ras2 in pancreatic cancer cell lines by immobilized polymerase chain reaction. BMC Biotechnol 3: 11).

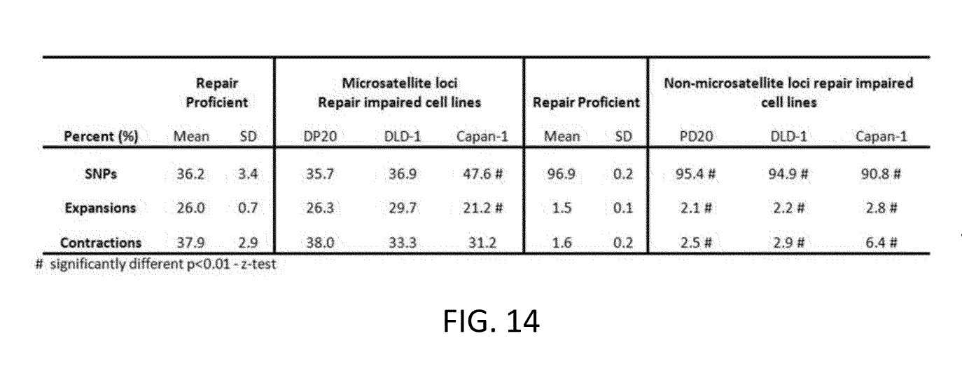

In contrast, there was an increase (5.5%) in heterozygotic loci in DLD-1 cells, which can potentially be attributed to increased mutation due to the MMR defects responsible for the MSI in DLD-1 cells. Surprisingly, haplotype distribution analysis at non-MST loci shows that DLD-1 cells, but not Capan-1 differ significantly from DNA repair proficient (1.8% compared to 1.2% for DLD-1 and Capan-1 respectively). This was unexpected because neither mutation mechanism (homologous recombination nor MMR) would necessarily be restricted to MST vs non-MST regions. A comparison of SNPs and INDELs in the DNA repair impaired cell lines showed Capan-1 cells significantly differed from the DNA repair proficient mean in the fraction of SNPs, with 47% and 91% for MST and non-MST loci respectively (the Table in FIG. 14). Conversely, DLD-1 and PD20 cells were not found to be different from DNA repair proficient cell lines. For the DNA repair proficient cells the mean fraction of loci with minor alleles was 5.1% with a SD of 0.4%. Capan-1 cells showed again, a greater susceptibility to mutation with a significant increase (6.2%) in the number of loci with minor alleles (the Table in FIG. 13). In contrast, PD20 and DLD-1 cells both show a significant decrease in loci with minor alleles, 3.1% and 3.2% respectively. This was surprising, particularly because the PD20 cells showed a decrease with respect to their corrected cell line PD20 RV:D2. Concordance of loci with minor alleles between the two related cell lines, PD20 and PD20 RV:D2, was 2.5% while discordance was 3.1%, which was significantly above chance (Pearson's X²). However, it was greater than the concordance between PD20 RV:D2 and MCF10A, which is to be expected since PD20 and PD20 RV:D2 are related strains (the Table in FIG. 11).

Because Capan-1 cells displayed the highest disparity in mutation rate from DNA repair proficient cell lines, including changes in SNP:INDEL ratios, the inventors decided to check the concordance of genotype and minor allele containing loci between them and PD20 RV:D2s (the Table in FIG. 11). Genotype concordance for the loci that were found in both samples, was over 97.3%, even higher than when the inventors compared PD20 RV:D2 with MCF10As. When comparing the loci with minor alleles ˜2% of the total had minor alleles in both samples (were concordant) however 12% were found to have minor alleles in only one samples, meaning discordance (the Table in FIG. 11). Although this is strikingly different, for the PD20 RV:D2 cells to MCF10A comparison, the concordance rate is still significantly greater than expected by chance. Very similar results were obtained when Capan-1 cells were compared to MCF10A cells. These results offer additional support the hypothesis that some MST loci are more susceptible to mutations than others.

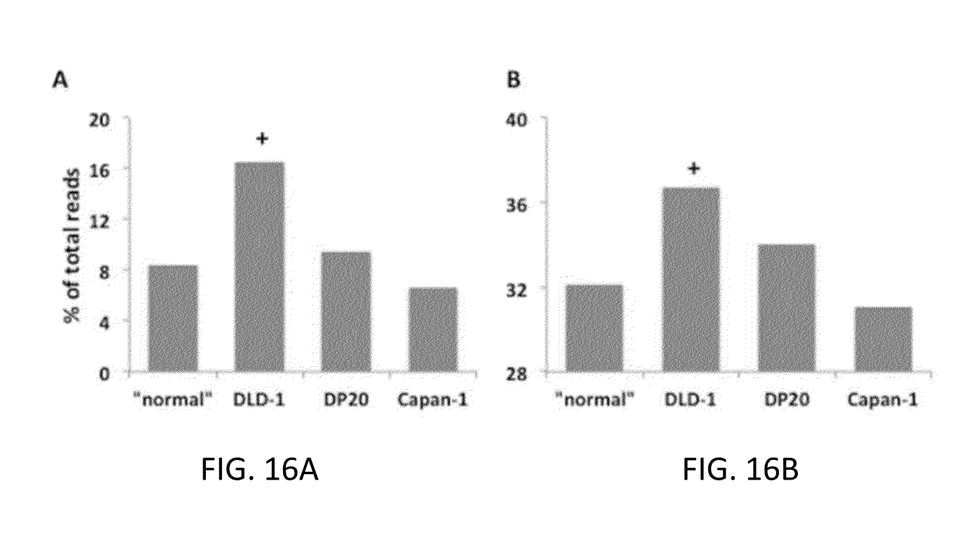

For DLD-1 cells, the increase in heterozygotic loci coupled with the significant reduction in the number of minor alleles is counterintuitive. This suggests the possibility of a proliferation of a small number of subpopulations. Two things are expected to occur: 1) an increase the average depth of reads that define the second allele and 2) an increase in the read depth supporting minor alleles without an increase in the number. To test this, the fraction of total reads covering the second allele regardless of haplotype and reads covering only minor alleles were compared. As depicted in FIGS. 16A and 16B, DLD-1 cells show greater than a 4% increase with respect to the DNA repair proficient average in the fractional coverage of the second allele and more than 8% increase (FIGS. 16A and 16B) for the percent coverage supporting minor alleles. Both were statistically significant. Neither Capan-1 nor PD20 were found to be different from the DNA repair proficient group for either of these parameters. These results suggest a population bottleneck where only a small number of distinct subpopulations are the predominant contributors of the reads captured by the sequencer.

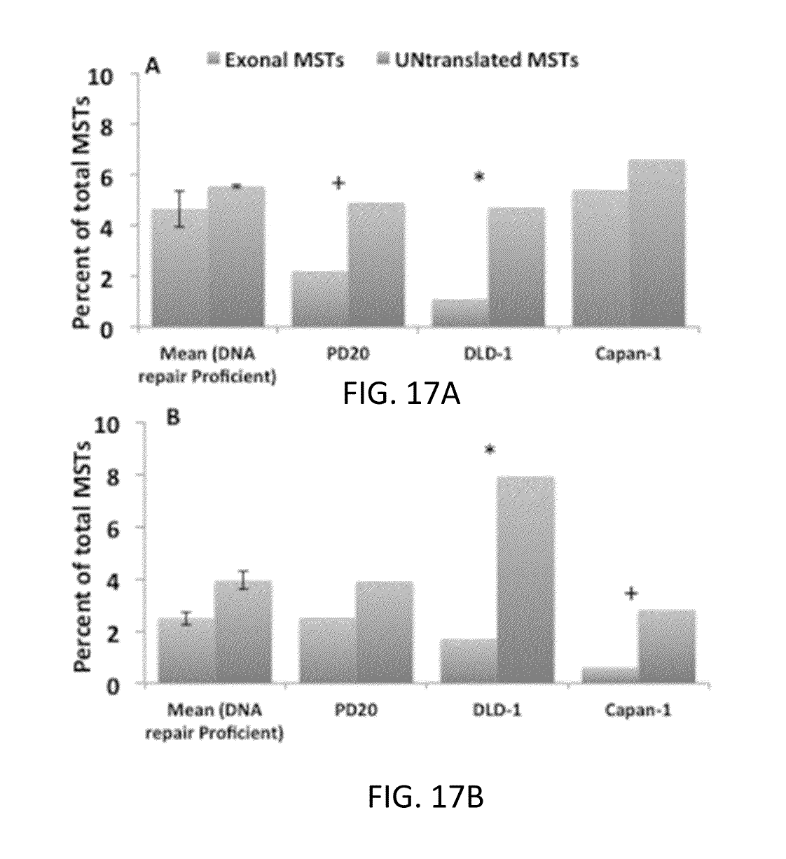

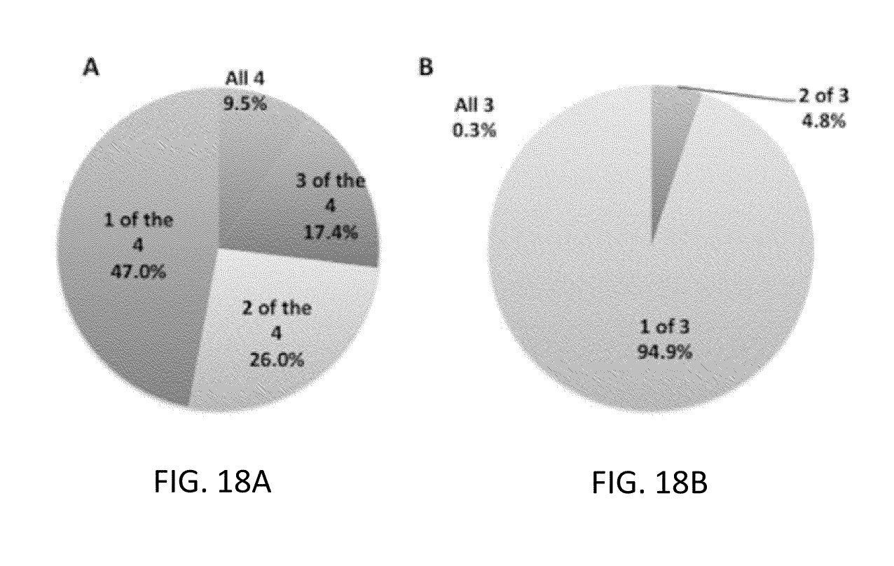

SMV in exons: MSTs are present ubiquitously throughout the genome and are found in over 16% of exons (Gemayel R, Vinces M D, Legendre M, Verstrepen K J (2010) Variable tandem repeats accelerate evolution of coding and regulatory sequences. Annu Rev Genet 44: 445-477). Although MST expansions or contractions in promoter and interexonal regions can affect transcription, mutations in exons are the most frequently implicated in downstream effects, consistent with exons being under significant selective pressure. An analysis of heterozygotic loci found that exons had significantly less heterozygotic loci, a reduction of over 1.2% compared to untranslated regions (2.4% and 3.8% respectively, FIG. 17A). However the difference in the fraction of loci with minor alleles in exons and untranslated regions was not significant (5.1% and 5.6%, FIG. 17B). In the previous sections the inventors showed that DLD-1 cells, a strain defective in MMR, was found, unexpectedly, to have a significant reduction in the number of MST loci with minor alleles and an increase in heterozygotic loci. Based on this comparison it appears that the results are due to the increased difference between translated and untranslated regions. As shown in FIG. 18A, the fraction of MST loci with minor alleles in exons is 1.1% (compared to 4.7% in untranslated regions) while the fraction of loci that are heterozygotic is 1.7%, compared to 7.9% in untranslated regions (FIG. 18B). These results further support hypothesis that DLD-1 cells have undergone a population bottleneck.

To determine the potential genetic implications of minor allele hot spots, the inventors focused on the analysis of genes affected, specifically they inspected genes containing MST loci found in exons that with 2 or more alleles that did not contribute to the haplotype (minor alleles). Of the 2603 genes whose exons harbor minor allele containing loci found in at least one of the 4 DNA repair proficient samples sequenced 47% were found to have 2 or more minor alleles in more than one sample and 9.5% were found in all 4 samples (FIG. 17A). A Genome Ontology (GO) analysis of the 247 genes harboring MSTs with multiple minor alleles in all 4 samples found only a borderline (p<0.01, the inventors use a lower p then 0.05 to compensate for the number of comparisons) significant enrichment of GOTERM categories that included transcription factors, regulators, repressors and DNA binding genes. In addition, there was no significant enrichment for any KEGG pathway categories or cataloged disorders. Conversely, of the ˜1100 minor allele harboring genes found in the DNA repair impaired cell lines, only 3 (0.27%) were found in all three cell lines while 95% are in only 1 of the three cell lines (FIG. 17B), which suggests this concordance pattern was primarily random. Further, no genes with multiallelic MSTs were found in all of the sequenced samples and only 18 were found in 6 of the 7 cell line samples. A KEGG pathway enrichment analysis of the minor allele harboring genes found in the DNA repair impaired cell lines suggests a pattern associated with various cancer pathways. Significant KEGG terms enriched were general cancer, colorectal cancer, myeloma, cervical cancer and cell adhesion (with p<0.001). Together, these results support the hypothesis that specific MST loci in repair proficient cells are more susceptible to somatic mutations but the genes associated with them are not associated with any specific categorized pathway. In contrast, for cells that have impairments in DNA repair pathways, somatic mutations in MSTs appear in higher frequency in loci that are specific to the DNA repair deficiency, and these mutations are implicated in disease, specifically cancer.

Discussion

Somatic mutation can lead to subpopulations of cells carrying mutated alleles. These are examined in cancers, as tumors can be considered to contain subpopulations of cells, i.e. the tissues are not genomically homogenous (Tang D G (2012) Understanding cancer stem cell heterogeneity and plasticity. Cell Res 22: 457-472; and Schor S L (1995) Fibroblast subpopulations as accelerators of tumor progression: the role of migration stimulating factor. EXS 74: 273-296). Tumors usually carry an allele or set of alleles that confirm their abnormal growth. These alleles, when detected in the tumor but not parent cells, can be the basis for important clinical treatment decisions (Hong S P, Min B S, Kim T I, Cheon J H, Kim N K, et al. (2012) The differential impact of microsatellite instability as a marker of prognosis and tumour response between colon cancer and rectal cancer. Eur J Cancer 48: 1235-1243; Hou Y, Song L, Zhu P, Zhang B, Tao Y, et al. (2012) Single-cell exome sequencing and monoclonal evolution of a JAK2-negative myeloproliferative neoplasm. Cell 148: 873-885; and Schor S L (1995) Fibroblast subpopulations as accelerators of tumor progression: the role of migration stimulating factor. EXS 74: 273-296). In cell populations with increased somatic mutation rates, like those with altered DNA repair capacity, there may be a concordant increase in subpopulation diversity. As a subpopulation propagates the mutations become more abundant, which becomes detectable in next-generation sequencing data (Schmitt M W, Kennedy S R, Salk J J, Fox E J, Hiatt J B, et al. (2012) Detection of ultra-rare mutations by next-generation sequencing. Proc Natl Acad Sci USA 109: 14508-14513; and Gundry M, Vijg J (2012) Direct mutation analysis by high-throughput sequencing: from germline to low-abundant, somatic variants. Mutat Res 729: 1-15). A major assumption of our analysis is that an increase in the number of alleles detected in next-generation sequencing data is reflective of an increase in cell subpopulations or somatic mutation present in the sequenced sample. In this Example the inventors evaluate allele frequencies at MSTs in various cell populations as a quantifiable indicator of variation.

The data presented here evaluate both the standard genotype and minor alleles that are present in next-generation data to establish a baseline for SMV in DNA repair proficient cells and compare this to cells with altered DNA repair capacity. There are several major objectives/findings from this analysis including (1) complimenting genomic analysis away of matched DNA samples with in-sample quantification of variation, (2) demonstrating that DNA repair proficient cells and those with different defects in DNA repair can have different SMV profiles that may be potential markers for these defects and (3) a quantitative measure of the fraction of loci that exhibit minor alleles may be reflective of subpopulations of cells with different genomic content, potentially those cells that may contribute to tumor formation. MST instability is important in the prognosis and selection of treatment for various cancers, and better, more accurate identification methods are always being sought (Xiao H, Yoon Y S, Hong S M, Roh S A, Cho D H, et al. (2013) Poorly differentiated colorectal cancers: correlation of microsatellite instability with clinicopathologic features and survival. Am J Clin Pathol 140: 341-347; and Hong S P, Min B S, Kim T I, Cheon J H, Kim N K, et al. (2012) The differential impact of microsatellite instability as a marker of prognosis and tumour response between colon cancer and rectal cancer. Eur J Cancer 48: 1235-1243).

These data demonstrate that the SNP:INDEL ratio at MSTs can be used to distinguish between different in-vivo mutational mechanisms and PCR amplified genomes. Both the WGA single cell sample and the Capan-1 cell line showed an increase in SNPs compared to INDELs at MST loci, however the fractions differed greatly. This is consistent with what was expected from both nucleotide mis-incorporation errors by polymerases (WGA single cell sample) and defects in DNA repair (Capan-1). Neither DLD-1 nor PD20 cells, which are defective in MMR and interstrand cross-link repair, respectively, had a significant alteration of the ratio of SNPs:INDELs at MST loci.

Capan-1 cells displayed a reduction of heterozygotic loci as compared to DNA repair proficient cell lines. This was expected since Capan-1 cells are a BRCA2− cells (impaired in homologous recombination) and have been shown to exhibit a loss of heterozygocity (Butz J, Wickstrom E, Edwards J (2003) Characterization of mutations and loss of heterozygosity of p53 and K-ras2 in pancreatic cancer cell lines by immobilized polymerase chain reaction. BMC Biotechnol 3: 11). However, the inventors' analysis also indicates a significant increase in the fraction of loci with minor alleles. This could be due to two reasons: 1) Capan-1 cells are a hypotriploid with over 35 structural rearrangements and with multiple chromosomal regions having more than three copies (Sirivatanauksorn V, Sirivatanauksorn Y, Gorman P A, Davidson J M, Sheer D, et al. (2001) Non-random chromosomal rearrangements in pancreatic cancer cell lines identified by spectral karyotyping. Int J Cancer 91: 350-358; and Grigorova M, Staines J M, Ozdag H, Caldas C, Edwards P A (2004) Possible causes of chromosome instability: comparison of chromosomal abnormalities in cancer cell lines with mutations in BRCA1, BRCA2, CHK2 and BUB1. Cytogenet Genome Res 104: 333-340). The minor alleles in Capan-1 cells can therefore be part of the genotype rather than somatic variation. Conversely, 2) Capan-1 cells have been reported to have an extremely high rate of INDELs and SNPs, significantly higher than expected from the hyperploidy (Barber L J, Rosa Rosa J M, Kozarewa I, Fenwick K, Assiotis I, et al. (2011) Comprehensive genomic analysis of a BRCA2 deficient human pancreatic cancer. PLoS One 6: e21639). The results shown here could be due to increased mutation rate shown with this cell line (Barber L J, Rosa J M, Kozarewa I, Fenwick K, Assiotis I, et al. (2011) Comprehensive genomic analysis of a BRCA2 deficient human pancreatic cancer. PLoS One 6: e21639) and further support general genomic instability in Capan-1 cells.

Unexpectedly, although DLD-1 cells are a MST unstable cell line, they did not display either of our predicted markers for increase in MST mutation rate: 1) an increase in the number of minor alleles, as was seen with Capan-1 cells, or 2) a decrease in the number heterozygotic loci and the number of minor alleles, as the inventors found in Capan-1 and PD20 cells (the Table in FIG. 14). Conversely, DLD-1 cells showed both a significant increase in the number of heterozygotic loci and a reduction in the fraction of loci with more than two alleles. Further, they displayed a great reduction in both the fraction of loci with minor alleles and heterozygotic loci in exons (conserved chromosomal regions). The inventors hypothesize that this is the result of defective MMR leading to an increase in mutations that have become fixed in the population. Alternatively, this may have resulted from a bottleneck in the growth of the cell population. If this was the case, the increase in heterozygotic loci allele may be a product of a limited set of surviving cell subpopulations. If a subpopulation with an un-repaired mutation, reached a sufficient proportion of the population due to the bottleneck it would generate sufficient reads for the locus to be mistakenly called heterozygotic. This point is reinforced by the significant increase in the portion of the total number of reads covering the second allele while the fraction of loci with minor alleles and the number of minor alleles per locus are decreased. This is important to note because it suggests that the inventors can not only distinguish between different mutational mechanisms using the minor alleles in next-generation sequencing, but may also be able to identify cells that have experienced a growth-limiting condition as they expand this work in the future.

The work presented here is a proof-of concept of an approach to assess somatic variation in MSTs using next-generation sequencing. Using this analysis the inventors were able to establish a SMV profile in DNA repair proficient cell lines which can be used to compare to cells with potential or known alterations in DNA repair capacity to begin to evaluate exome sequenced samples without requiring a matched genomic sample as baseline. Since somatic variation is part of genomic stability this approach might be used as an addition to current MST instability criteria.

The present invention has been described with reference to particular embodiments having various features. In light of the disclosure provided above, it can be apparent to those skilled in the art that various modifications and variations can be made in the practice of the present invention without departing from the scope or spirit of the invention. One skilled in the art can recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention can be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. A method of detecting a pathological feature in a subject, comprising:
taking a biological sample, wherein the biological sample comprises one or more cells;
subjecting the biological sample to DNA sequencing to determine one or more somatic microsatellite variability measurements in the biological sample; and
statistically comparing the one or more somatic microsatellite variability measurements in the biological sample with one or more somatic microsatellite variability measurements of one or more reference samples to identify one or more pathological features in the one or more cells of the subject;
wherein the one or more somatic microsatellite variability measurements are selected from the group consisting of percentage of SNPs, percentage of expansions, percentage of contractions, ratio of expansions and contractions to SNPs, percentage of heterozygotic loci, percentage of homozygotic loci, and percentage of loci with minor alleles.

2. The method of claim 1, wherein the biological sample is taken from a cell line.

3. The method of claim 1, wherein the biological sample is taken from a subject.

4. The method of claim 1, wherein the one or more pathological features are one or more deficient DNA repair pathways.

5. The method of claim 1, wherein the one or more reference samples are obtained from one or more cells with proficient DNA repair capabilities.

6. The method of claim 1, wherein the one or more reference samples are obtained from one or more cells with one or more deficient DNA repair pathways.

7. The method of claim 4, wherein the one or more deficient DNA repair pathways include homologous recombination, non-homologous end joining, DNA mismatch repair, base excision repair, nucleotide excision repair, and crosslink repair.

8. The method of claim 6, wherein the one or more deficient DNA repair pathways include homologous recombination, non-homologous end joining, DNA mismatch repair, base excision repair, nucleotide excision repair, and crosslink repair.

9. The method of claim 3, wherein the one or more pathological features are indicative of a genetic syndrome in the subject.

10. The method of claim 9, wherein the genetic syndrome is selected from the group consisting of Bloom Syndrome, Rothmund-Thomson, Fanconi Anemia, Werner Syndrome, BRCA1/2 mutation, Lynch Syndrome, Cockayne Syndrome, Xeroderma Pigmetnosum, and Trichothiodystropia.

11. The method of claim 3, wherein the one or more pathological features are indicative of a cancer in the subject.

12. The method of claim 3, wherein the one or more pathological features are indicative of the aggressiveness, stage, or grade of cancer in the subject.

13. The method of claim 1, wherein DNA sequencing is performed on the genome or the exome of the one or more cells.

14. The method of claim 5, wherein somatic microsatellite variability measurements in one or more reference samples representing proficient DNA repair capability is used to establish a baseline somatic microsatellite variability profile.

15. The method of claim 14, wherein a statistical comparison of somatic microsatellite variability measurements of the biological sample with the baseline somatic microsatellite variability profile may indicate altered DNA repair capability.

16. The method of claim 6, wherein a statistical comparison of somatic microsatellite variability measurements of the biological sample with somatic microsatellite variability measurements of one or more reference samples representing one or more deficient DNA repair pathways may indicate a deficiency in a specific DNA repair pathway in the biological sample.

17. A method of treating a patient, comprising:
taking a clinical sample from a patient, wherein the clinical sample comprises one or more cells;
subjecting the clinical sample to DNA sequencing to determine one or more somatic variability measurements in the clinical sample; and
statistically comparing the one or more somatic variability measurements in the clinical sample with one or more somatic variability measurements of one or more reference samples to interpret an amount of genomic instability in the clinical sample; and
making a treatment decision based on the interpretation;
wherein the one or more somatic variability measurements are selected from the group consisting of percentage of SNPs, percentage of expansions, percentage of contractions, ratio of expansions and contractions to SNPs, percentage of heterozygotic loci, percentage of homozygotic loci, and percentage of loci with minor alleles.

18. The method of claim 17, wherein the clinical sample is a biopsy of a cancer.

19. The method of claim 18, wherein the cancer is colon cancer.

20. The method of claim 17, wherein the clinical sample is blood and wherein the treatment decision concerns predicting risk of cancer, or other disease of the aged, or cardiac disease, or neurological disease.
